SEQUENCE LISTING (26 sequences)

SEQ ID NO: 1 - DNA, 21 bp, Mus musculus
  source 1..21: mol_type = other DNA
ctgagaaacggctaccacatc

SEQ ID NO: 2 - DNA, 22 bp, Mus musculus
  source 1..22: mol_type = other DNA
gcctcgaaagagtcctgtattg

SEQ ID NO: 3 - DNA, 20 bp, Mus musculus
  source 1..20: mol_type = other DNA
atggcctccctctcatcagt

SEQ ID NO: 4 - DNA, 20 bp, Mus musculus
  source 1..20: mol_type = other DNA
cttggtggtttgctacgacg

SEQ ID NO: 5 - DNA, 22 bp, Mus musculus
  source 1..22: mol_type = other DNA
cactgcgatcattctcgtcctg

SEQ ID NO: 6 - DNA, 22 bp, Mus musculus
  source 1..22: mol_type = other DNA
gactgtgtacccttacctccga

SEQ ID NO: 7 - DNA, 20 bp, Mus musculus
  source 1..20: mol_type = other DNA
gttctctgggaaatcgtgga

SEQ ID NO: 8 - DNA, 20 bp, Mus musculus
  source 1..20: mol_type = other DNA
gcattggaaattggggtagg

SEQ ID NO: 9 - DNA, 20 bp, Mus musculus
  source 1..20: mol_type = other DNA
agccaactctcactgaagcc

SEQ ID NO: 10 - DNA, 20 bp, Mus musculus
  source 1..20: mol_type = other DNA
ggacccattccttcttgggg

SEQ ID NO: 11 - DNA, 18 bp, Mus musculus
  source 1..18: mol_type = other DNA
ccgcagtcactccaggat

SEQ ID NO: 12 - DNA, 18 bp, Mus musculus
  source 1..18: mol_type = other DNA
tgcagtctgcccagttca

SEQ ID NO: 13 - DNA, 20 bp, Mus musculus
  source 1..20: mol_type = other DNA
agctcaccagaccctgagaa

SEQ ID NO: 14 - DNA, 20 bp, Mus musculus
  source 1..20: mol_type = other DNA
tcccagcaatcgttaccttc

SEQ ID NO: 15 - DNA, 21 bp, Mus musculus
  source 1..21: mol_type = other DNA
gtcaacaagtggtggcgtgtg

SEQ ID NO: 16 - DNA, 22 bp, Mus musculus
  source 1..22: mol_type = other DNA
gcagcaaaggcttctgggataa

SEQ ID NO: 17 - DNA, 20 bp, Mus musculus
  source 1..20: mol_type = other DNA
gtgcgcaggtacaagaaact

SEQ ID NO: 18 - DNA, 22 bp, Mus musculus
  source 1..22: mol_type = other DNA
ccaggttctcaatgtacatcga

SEQ ID NO: 19 - DNA, 21 bp, Mus musculus
  source 1..21: mol_type = other DNA
gcggagtggaaacttttgtcc

SEQ ID NO: 20 - DNA, 23 bp, Mus musculus
  source 1..23: mol_type = other DNA
gggaagcgtgtacttatccttct

SEQ ID NO: 21 - DNA, 19 bp, Mus musculus
  source 1..19: mol_type = other DNA
cggatgagctttgtgaagg

SEQ ID NO: 22 - DNA, 18 bp, Mus musculus
  source 1..18: mol_type = other DNA
gggtgctcgtgacatcct

SEQ ID NO: 23 - DNA, 20 bp, Mus musculus
  source 1..20: mol_type = other DNA
gccgggaatgatgagaacta

SEQ ID NO: 24 - DNA, 20 bp, Mus musculus
  source 1..20: mol_type = other DNA
ggtgaaactcttgcctcgtc

SEQ ID NO: 25 - DNA, 18 bp, Mus musculus
  source 1..18: mol_type = other DNA
tgagcgacacggacaaga

SEQ ID NO: 26 - DNA, 20 bp, Mus musculus
  source 1..20: mol_type = other DNA
ggcctggtagttgttgtgag

(19) United States
(12) Patent Application Publication
(10) Pub. No.: US 2025/0154468 A1
(43) Pub. Date: May 15, 2025

(54) METHOD FOR INDUCING DEDIFFERENTIATION OF ADIPOCYTES

(71) Applicant: City University of Hong Kong, Hong Kong (HK)

(72) Inventors: Liang ZHANG, Hong Kong (HK); Guopan LIU, Hong Kong (HK); Yilin PAN, Tai’an City (CN); Ying WANG, Hong Kong (HK)

(21) Appl. No.: 18/619,199

(22) Filed: Mar. 28, 2024

Related U.S. Application Data
(60) Provisional application No. 63/598,153, filed on Nov. 13, 2023.

(51) Int. Cl. C12N 5/077

(52) U.S. Cl. CPC C12N 5/0654; C12N 5/0655; C12N 2501/148; C12N 2501/21; C12N 2501/2306; C12N 2501/415; C12N 2501/727; C12N 2506/13

(57) ABSTRACT

The present invention investigates the effects of hypertonicity on adipocytes, revealing its role in inducing the release of mitochondrial extracellular vesicles (MEVs). This release subsequently enhances the secretion of TNF-α, a pro-inflammatory cytokine crucial during stress responses, thereby activating the Wnt/β-catenin signaling pathway responsible for adipocyte dedifferentiation. Through the present method, hypertonicity induces MEVs release and TNF-α secretion, ultimately modulating Wnt/β-catenin signaling and promoting adipocyte dedifferentiation, offering promising therapeutic implications.

### CROSS-REFERENCE TO RELATED APPLICATIONS

The present application claims the priorities from the U.S. provisional patent application Ser. No. 63/598,153 filed Nov. 13, 2023, and the disclosure of which is incorporated herein by reference in its entirety.

### REFERENCE TO SEQUENCE DISCLOSURE

The sequence listing file under the file name “P2989US01_Sequence_listing.xml” submitted in ST.26 XML file format with a file size of 23.1 KB created on Apr. 1, 2024 is incorporated herein by reference.

### FIELD OF THE INVENTION

The present invention pertains to the field of cell biology, biochemistry, protein analysis, biomedical research, and potentially drug development.

### BACKGROUND OF THE INVENTION

With a mesenchymal origin, adipocytes are recognized as an essential energy storage entity as well as a critical endocrine source that regulates systemic metabolism. Despite the essential physiological functions, hypertrophy and hyperproliferation of adipose tissues lead to obesity, which is a major risk factor for a series of diseases including dyslipidemia, cardiovascular disease, type 2 diabetes and cancer. At the cellular level, obesity is underlined by the deregulation of adipogenesis, the differentiation of committed pre-adipocytes into mature adipocytes. Interestingly, adipocytes do not remain at a static stage of terminal differentiation after adipogenesis. Instead, mature adipocytes display a high degree of plasticity and can be reverted to a multipotent progenitor-like state. This is typically termed adipocyte dedifferentiation and has been broadly observed in vitro and in vivo. For example, during late pregnancy and lactation, mammary adipocytes lose lipid droplets and dedifferentiate into fibroblast-like preadipocytes that later re-differentiate into adipocytes after lactation[1]. Moreover, recent studies observed adipocytes dedifferentiation in the development of different malignant tumors, which was associated with the activation of Wnt signaling [2, 3] and Notch signaling [4]. A better understanding of the signaling routes that regulate the differentiation/dedifferentiation balance of adipocytes holds the key to effectively preventing and treating obesity and related diseases.

The mechanisms that regulate adipogenesis have been extensively investigated. However, much less is known about the molecular mechanisms underlying adipocyte dedifferentiation. Generally, pro- and anti-adipogenic genes/pathways play opposite roles in adipocyte dedifferentiation. For example, C/EBPβ and PPARγ are key pro-adipogenic genes and are typically suppressed to allow adipocyte dedifferentiation. As a PPARγ agonist, rosiglitazone could block adipocyte dedifferentiation and tumor development in the Notch-driven liposarcoma mouse model[4]. In contrast, canonical Wnt/β-catenin signaling ligands are recognized as anti-adipogenic factors and have been identified as enhancers of dedifferentiation. While overexpression of Wnt1 gene inhibited adipogenic differentiation of progenitor cells, exogenous application of Wnt3a could induce dedifferentiation of both mouse 3T3-L1 and human adipocytes[2, 5-6]. In addition to Wnt signaling, Notch, TGF-β and TNF-α signaling have all been reported to suppress adipogenesis and promote adipocyte dedifferentiation in various contexts[4, 6-8]. The detailed mechanism of how different signaling pathways cooperatively regulate adipocyte differentiation warrants further investigation.

Several methods have been established to induce adipocyte dedifferentiation, facilitating the investigation of molecular and cellular mechanisms. The “ceiling culture” of mature adipocytes was originally reported to induce the loss of lipid droplets and the emergence of progenitor-like cells, namely dedifferentiated fat cells (DFAT cells)[9]. Activation of TGF-β1 signaling and different collagens can regulate DFAT cells of the ceiling culture system. Recently, physical changes in microenvironments, including matrix stiffening, increased compressive force, and elevated osmotic pressure, have been shown to affect the cell fates and adipocyte dedifferentiation[10-11]. Accordingly, hypertonic treatment has been shown to induce osmotic stress and promote adipocyte dedifferentiation. Given the ample cellular abundance and the simple operation, hypertonicity-induced DFAT holds a strong promise to transform regenerative medicine. Mechanistically, activation of the anti-adipogenic Wnt/β-catenin signaling was observed during the osmotic reprogramming of adipocytes.

However, it remains elusive how hypertonicity activates the Wnt/β-catenin signaling and drives the dedifferentiation. This invention addresses this need.

### SUMMARY OF THE INVENTION

The present invention is aimed to elucidate the molecular mechanisms that underlie osmotic induction of adipocyte reprogramming.

In a first aspect, the present invention provides a method for inducing dedifferentiation of adipocytes, including subjecting one or more adipocytes to a hypertonic solution; and inducing release of mitochondrial extracellular vesicles (MEVs) from the one or more adipocytes to an extracellular environment. The released MEVs enhance the secretion of a series of inflammatory genes from the one or more adipocytes, and the series of inflammatory genes activates the Wnt/β-catenin signaling pathway, thereby driving adipocyte dedifferentiation.

In an aspect, the series of inflammatory genes include TNF-α, IL-6, RIP1, CEBPA, and MCP-1.

Preferably, the series of inflammatory genes include TNF-α and IL-6.

In an aspect, the one or more adipocytes include primarily isolated adipocytes, adipocytes derived from induced pluripotent stem cells (iPSCs) or embryonic stem cells (ESCs), adipocytes from different anatomical locations.

In another aspect, the one or more adipocytes include 3T3-L1 or stromal vascular fraction (SVF)-derived adipocytes.

In an aspect, the adipocyte count of the one or more adipocytes is decreased by at least 3.5 times following hypertonic treatment.

In another aspect, the hypertonic treatment results in reductions of 40-60% in the expression of adipogenic markers comprising C/EBPβ, PPAR-γ, and adiponectin.

In a further aspect, the hypertonic treatment results in increase of 10-20% in the expression of surface markers associated with dedifferentiated adipocytes.

In an aspect, the hypertonic solution is formulated to have a hypertonic pressure compared to the surrounding environment, and the hypertonic solution comprises a culture medium and 2% PEG 300. The culture medium may be DMEM. The hypertonic solution further contains an additive including a preservative, a stabilizer, or a medication.

In an aspect, the one or more adipocytes treated with the hypertonic solution exhibit a capability for osteogenic and chondrogenic re-differentiation.

In another aspect, the method further includes adding a small molecule compound to mitigate apoptosis of the one or more adipocytes induced by hypertonic treatment. The small molecule compound comprises. 2-amino-4-(3,4-(methylenedioxy)benzylamino)-6-(3-methoxyphenyl)pyrimidine.

In a further aspect, the one or more adipocytes exhibit a decrease in the expression of adipogenic genes, coupled with concurrent elevations in the expression of marker genes. The adipogenic genes comprise C/EBPβ, PPAR-γ, and adiponectin. The marker genes comprise Smad 9, Esrrb, and Sox2.

In a further aspect, the one or more adipocytes exhibit a capability for osteogenic and chondrogenic re-differentiation.

The present invention establishes a robust cellular model, where high-osmolarity efficiently induces dedifferentiation of 3T3-L1 and stromal vascular fraction (SVF)-derived adipocytes. Then, proteomic profiling of extracellular vesicles (EVs) released from the dedifferentiating adipocytes receiving hypertonic treatment is conducted. The result reveals that hypertonicity prompts the adipocytes to release mitochondria via EVs, which in turn enhances the secretion of the TNF-α cytokine during the stress response. Importantly, hypertonicity-induced EVs and TNF-α play a critical role in activating the Wnt/β-catenin signaling pathway that drives adipocyte dedifferentiation. Overall, the present invention defines a novel mitochondria-TNF-α axis that underlies the osmotic regulation of Wnt/β-catenin signaling and adipocyte dedifferentiation.

Compared to existing technologies, the present method offers a more effective and controllable approach for generating mitochondrial extracellular vesicles (MEVs). Through the application of hypertonic shock to adipocytes, it enhances the controlled release of MEVs containing mitochondrial components. The present invention provides a platform for drug development and screening, utilizing the MEVs to study cellular communication, signaling pathways, and drug responses. In summary, it offers a novel approach to enhance MEVs production through hypertonicity, enabling applications in disease diagnosis, therapeutics, regenerative medicine, tissue engineering.

### BRIEF DESCRIPTION OF THE DRAWINGS

Embodiments of the invention are described in more details hereinafter with reference to the drawings, in which:

FIG. 1A shows a diagram showing the experimental workflow. FIG. 1B shows representative images of adipocytes in the hypertonic culture for the indicated time. Scale bar: 100 μm. FIG. 1C depicts quantification of adipocytes losing clear lipid droplets over time in the indicated culture conditions. Error bars represent SD (n=3 independent experiments). FIG. 1D shows images and quantification of Oil Red O staining of adipocytes following indicated treatments for seven days. FIG. 1E depicts box blots showing the aspect ratio of indicated adipocytes (n=200 cells) following control or hypertonic treatments of 4 days. FIG. 1F depicts RT-qPCR analysis of the expression of C/EBPβ, PPARγ, and adiponectin in isotonic and hypertonic treated adipocytes;

FIG. 2A shows immunostaining of CD13 in control and hypertonic treated 3T3-L1 adipocytes. Percentage of cells with positive CD13 labeling are quantified in (B) Scale bar: 20 μm. FIG. 2B shows immunostaining of Endoglin in control and hypertonic treated 3T3-L1 adipocytes. Percentage of cells with positive Endoglin labeling are quantified in (D) Scale bar: 20 μm;

FIG. 3A shows representative images of alkaline phosphatase (ALP) staining showing the osteogenic potential of the de-differentiated 3T3-L1 adipocytes. FIG. 3B depicts RT-qPCR analysis of the expression of Runx2 and ALP following osteogenic induction of isotonic and hypertonic treatment of 3T3-L1 adipocytes. FIG. 3C depicts RT-qPCR analysis of the expression of Smad3, Col2a1 and Sox9 following chondrogenic induction of isotonic and hypertonic treatment of 3T3-L1 adipocytes;

FIG. 4A shows EVs from isotonic control and hypertonic culture of 3T3L1 adipocytes are subject to quantitative proteomic analysis. FIG. 4B shows a Venn diagram compares the proteins of control and hypertonic EVs from adipocytes. The right graph depicts the GO-term analysis of the 25 hypertonic-unique proteins. FIG. 4C depicts scatter plot showing the differential analysis of the EVs proteome between the control and hypertonic pressure. Sloping dotted lines demarcate tenfold difference in the abundance. FIG. 4D depicts GO-term analysis of the up-regulated proteins in the hypertonic EVs. The top five cellular components are shown. FIGS. 4E-4F shows the 3T3-L1 adipocytes receiving indicated treatments for 8 h were stained using MitoTracker Green and DAPI. Relative intensities of the green channel were quantified. Scale bars, 10 μm. FIG. 4G shows representative transmission electron microscopy (TEM) images of hypertonic EVs from 3T3-L1 adipocytes. Arrows indicate inclusions of mitochondria fragments. FIG. 4H shows cell lysates and EVs of 3T3-L1 adipocytes following indicated treatments are subject to immunoblot analyses of the indicated proteins, respectively;

FIG. 5 shows the levels of NDUFA9 and CD81 assessed by Western blot analysis in 3T3-L1 adipocytes after the indicated treatments, including isotonic+DMSO, isotonic+GW4869, hypertonic+DMSO, or hypertonic+GW4869;

FIG. 6A shows EVs from control or hypertonic cultures of 3T3-L1 adipocytes were used to treat naïve 3T3-L1 adipocytes in isotonic culture. FIG. 6B depicts RT-qPCR analysis of the expression of C/EBPβ, PPARγ, and adiponectin in 3T3-L1 adipocytes treated with EVs isolated from the indicated cultures. FIG. 6C depicts RT-qPCR analysis of the expression of TNF-α and IL-6 in 3T3-L1 adipocytes treated with EVs isolated from the adipocytes from the indicated cultures;

FIG. 7A depicts RT-qPCR analysis of the expression of RIP1, CEBPA and MCP-1 in 3T3-L1 adipocytes treated with EVs isolated from the adipocytes from the indicated cultures. FIG. 7B depicts RT-qPCR analysis of the expression of RIP1, CEBPA and MCP-1 in 3T3-L1 adipocytes receiving indicated treatments;

FIG. 8A depicts RT-qPCR analysis of the expression of TNF-α in 3T3-L1 adipocytes receiving EVs from the indicated treatments. FIG. 8B depicts RT-qPCR analysis of the expression of TNF-α and IL-6 in 3T3-L1 adipocytes receiving indicated treatments;

FIG. 9A depicts qPCR analysis of TNF-α related inflammatory factor in 3T3-L1 adipocytes receiving indicated treatments for 2 hours. FIG. 9B depicts ELISA analysis of the protein levels of TNF-α in the media of 3T3-L1 adipocytes receiving indicated treatments for 8 hours;

FIG. 10A shows RT-qPCR analysis of the expression of Smad9, Esrrb and Sox2 in adipocytes receiving the indicated treatments. FIG. 10B shows total cell lysates from 3T3-L1 adipocytes treated by EVs from isotonic or hypertonic adipocyte cultures subject to western blot analyses of indicated proteins. FIG. 10C shows total cell lysates from 3T3-L1 adipocytes in indicated culture conditions subject to western blot analyses of indicated proteins. FIG. 10D shows representative images of immunostaining of active β-catenin in 3T3-L1 adipocytes following control or hypertonic treatment. FIG. 10E depicts quantification of the intensity of active β-catenin staining in the nucleus of adipocytes following control or hypertonic treatment for 24 hours. FIG. 10F depicts 3T3-L1 adipocytes were treated with the indicated conditions for 24 hours and the cell lysates subject to western blot analysis of indicated proteins. FIG. 10G shows representative results of annexin V/PI flow cytometry analysis of adipocytes receiving indicated treatments. FIG. 10H depicts bar graphs of the proportions of cells in early-phase apoptosis in adipocytes receiving indicated treatment;

FIG. 11A shows representative images of adipocytes treated by BML-284 for the indicated time. Scale bar, 100 μm. FIG. 11B depicts quantification of adipocytes losing all their clear lipid droplets during seven days of control or BML-284 culture. Error bars represent SD (n=3, independent repeats). FIG. 11C shows images and quantification of Oil Red O show adipocytes losing all their clear lipid droplets following indicated treatments for seven days. FIG. 11D depicts box blots depict the aspect ratio of indicated adipocytes (n=200 cells) following control or BML-284 treatments. FIG. 11E depicts RT-qPCR analysis of the expression of C/EBPβ, PPARγ, and adiponectin in control or BML-284 treated adipocytes. FIG. 11F depicts RT-qPCR analysis of the expression of pluripotency markers Smad9, Esrrb and Sox2 in control or BML-284 treated adipocytes;

FIG. 12A depict immunostaining of CD13 in control and BML 284 treated 3T3-L1 adipocytes, and percentage of cells with positive CD13 labeling. Scale bar: 20 μm. FIG. 12B depicts immunostaining of Endoglin in control and BML 284 treated 3T3-L1 adipocytes, and percentage of cells with positive Endoglin labeling. Scale bar: 20 μm;

FIG. 13A depicts RT-qPCR analysis of the expression of Smad3, Col2a1 and Sox9 of isotonic and BML-284 treated adipocytes following chondrogenic induction. FIG. 13B shows representative images of alkaline phosphatase (ALP) staining showing the osteogenic potential of the de-differentiated 3T3-L1 adipocytes following control or BML-284 treatments, and RT-qPCR analysis of the expression of ALP following osteogenic induction of control- or BML-284-treated 3T3-L1 adipocytes. FIG. 13C depicts representative results of annexin V/PI flow cytometry analysis of adipocytes receiving indicated treatments. Bar graphs depict the proportions of cells in early-phase apoptosis in adipocytes receiving indicated treatments; and

FIG. 14 shows a working model of adipocyte dedifferentiation induced by hypertonicity.

### DETAILED DESCRIPTION

### Definitions

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

MEVs function in an autocrine fashion to enhance the pro-inflammatory TNF-α signaling that activates β-catenin and drives adipocyte dedifferentiation. Adipocytes have the potential to dedifferentiate into the multipotent progenitor state.

Recent studies demonstrated that hypertonicity could induce adipocyte dedifferentiation, representing an appealing way to produce regenerative toolsets. Alleviating mitochondrial stress inhibits MEVs release and hypertonicity-induced adipocyte dedifferentiation. However, it remains elusive about the molecular mechanism that underlies the hypertonicity-induced reprogramming of adipocytes.

Accordingly, the present invention provides a method for inducing dedifferentiation of adipocytes, including subjecting one or more adipocytes to a hypertonic solution; and inducing release of mitochondrial extracellular vesicles (MEVs) from the one or more adipocytes to an extracellular environment. The released MEVs enhance the secretion of a series of inflammatory genes from the one or more adipocytes, and the series of inflammatory genes activates the Wnt/β-catenin signaling pathway, thereby driving adipocyte dedifferentiation. The present invention defines a novel signaling axis that promotes the multipotent dedifferentiation of adipocytes.

In one embodiment, the adipocytes may include primarily isolated adipocytes, adipocytes derived from induced pluripotent stem cells (iPSCs) or embryonic stem cells (ESCs), adipocytes from different anatomical locations (e.g., subcutaneous adipocytes, visceral adipocytes, or brown adipocytes).

Preferably, the adipocytes may be 3T3-L1 cells or stromal vascular fraction (SVF)-derived adipocytes.

The present invention delves deep into the mechanism underlying the reprogramming of adipocytes induced by hypertonicity. The hypertonicity induces adipocytes to release mitochondrial extracellular vesicles (MEVs), which, in turn, function in an autocrine manner to enhance the secretion of TNF-α as a pro-inflammatory cytokine during the stress response.

Hypertonic solutions contain a higher concentration of solutes compared to the surrounding environment or another solution. The solution may include a culture medium and 2% PEG 300 to help maintain a stable pH level, an additive.

In one embodiment, the additive may include preservatives, stabilizers, or medications.

MEVs exhibit potential in modulating cellular metabolism, enhancing mitochondrial function, and influencing diverse cellular pathways. Specifically:
(1) Modulation of cellular metabolism: MEVs encapsulate enzymes and metabolic regulators pivotal for cellular metabolism. Upon internalization by recipient cells, MEVs exert influence over metabolic pathways such as glycolysis, oxidative phosphorylation, and fatty acid oxidation.
(2) Enhancement of mitochondrial function: MEVs commonly harbor components essential for optimizing mitochondrial function. These components may include mitochondrial DNA (mtDNA), mitochondrial RNA (mtRNA), and proteins involved in mitochondrial biogenesis, fusion-fission dynamics, and electron transport chain activity. Delivery of such constituents to recipient cells enhances mitochondrial integrity, augments ATP production, and overall improves cellular energy metabolism.
(3) Influence on cellular signaling pathways: MEVs serve as potent modulators of cellular signaling pathways by transporting signaling molecules such as growth factors, cytokines, and microRNAs.
(4) Therapeutic potential: Engineered MEVs or cargo molecules derived from MEVs can be tailored for targeted delivery of therapeutic agents, genetic material, or metabolic modulators to specific cell types or tissues. Moreover, MEVs isolated from stem cells or alternative sources may harbor regenerative properties, presenting opportunities for innovative tissue repair or regeneration strategies.

MEVs may activate the Wnt/β-catenin signaling pathway, which plays a crucial role in regulating adipogenesis, adipocyte differentiation, and metabolic homeostasis. TNF-α is shown to be essential for the activation of the Wnt/β-catenin signaling pathway. Activation of this pathway can lead to changes in gene expression patterns that promote adipocyte dedifferentiation and alter cellular metabolism. When Wnt proteins bind to their receptors on the cell membrane, it initiates the Wnt/β-catenin signaling pathway. This leads to stabilization of β-catenin protein, allowing it to translocate into the nucleus where it interacts with transcription factors, thereby initiating the transcription of specific genes. In adipocytes, activation of the Wnt/β-catenin signaling pathway can result in changes in the expression patterns of various genes, including those involved in adipocyte differentiation and metabolism. TNF-α has been shown to be essential for the activation of the Wnt/β-catenin signaling pathway. This is because TNF-α can promote the stability of β-catenin, thereby enhancing its accumulation in the nucleus and transcriptional activity. Consequently, TNF-α can regulate adipocyte dedifferentiation and cellular metabolism by modulating the activity of this signaling pathway.

In addition to the activation of the Wnt/β-catenin signaling pathway, MEVs may also be involved in several other signaling pathways that contribute to the reprogramming of adipocytes induced by hypertonicity. For example, MEVs may influence the AMP-activated protein kinase (AMPK) pathway, a master regulator of cellular energy homeostasis. Activation of AMPK can lead to increased fatty acid oxidation, glucose uptake, and mitochondrial biogenesis, thereby impacting adipocyte metabolism and function. MEVs may influence activate the NF-κB signaling pathway, a key regulator of inflammation and immune responses. In addition, MEVs may activate the MAPK/ERK signaling pathway, which regulates various cellular processes including cell proliferation, differentiation, and inflammation. Activation of this pathway can modulate adipocyte phenotype and function, potentially contributing to hypertonicity-induced adipocyte reprogramming. Furthermore, MEVs may also modulate the PI3K/Akt signaling pathway, which regulates cell survival, proliferation, and metabolism. Activation of Akt signaling can promote adipocyte dedifferentiation and alter cellular metabolism in response to hypertonicity-induced stress.

It is crucial to underscore a potential limitation of hypertonic treatment, namely its propensity to induce apoptosis. However, the present invention offers an effective solution to this issue by simultaneously promoting adipocyte dedifferentiation and mitigating apoptosis through direct activation of the Wnt/β-catenin signaling pathway using BML-284. BML-284 is known to directly activate the Wnt/β-catenin signaling pathway.

BML-284 is a small molecule compound that functions as an inhibitor of glycogen synthase kinase 3 beta (GSK-30). GSK-30 is a serine/threonine kinase that plays a crucial role in various cellular processes, including glycogen metabolism, cell proliferation, apoptosis, and gene transcription regulation. In the context of the Wnt/β-catenin signaling pathway, GSK-30 phosphorylates β-catenin, targeting it for degradation by the proteasome, thereby keeping the pathway inactive. It functions by inhibiting the activity of GSK-30, a kinase that normally phosphorylates β-catenin, targeting it for degradation. By inhibiting GSK-30, BML-284 prevents β-catenin degradation, leading to its stabilization and accumulation in the cytoplasm. This stabilized β-catenin then translocates into the nucleus, where it interacts with transcription factors of the T-cell factor/lymphoid enhancer factor (TCF/LEF) family to activate the transcription of Wnt target genes. These target genes are involved in various cellular processes, including cell survival, proliferation, and differentiation. Therefore, by directly activating the Wnt/β-catenin signaling pathway, BML-284 can counteract the apoptotic effects induced by hypertonic treatment. This activation promotes cell survival and can potentially enhance the dedifferentiation process of adipocytes, providing a dual benefit in the context of hypertonicity-induced cellular responses.

In summary, the present invention has uncovered the mechanisms underlying the multipotent dedifferentiation of adipocytes, presenting great promise for applications in regenerative medicine. A novel signaling axis has been identified, involving high osmolarity-induced mitochondrial stress, EV-mediated mitochondrial ejection, TNF-α secretion, and Wnt/β-catenin activation, driving adipocyte dedifferentiation. Furthermore, the present invention demonstrates that direct activation of Wnt/β-catenin signaling using BML-284 efficiently induces adipocyte differentiation while overcoming the apoptotic effects of hypertonic treatment.

### EXAMPLE

### Example 1

### Induction of Adipocyte Dedifferentiation by Hypertonicity in 3T3-L1 Cells

Recent studies demonstrated that the physical compression induced by hypertonic treatment could lead to dedifferentiation of adipocytes derived from primary sources [11]. However, this phenomenon has not been recapitulated in cell line models.

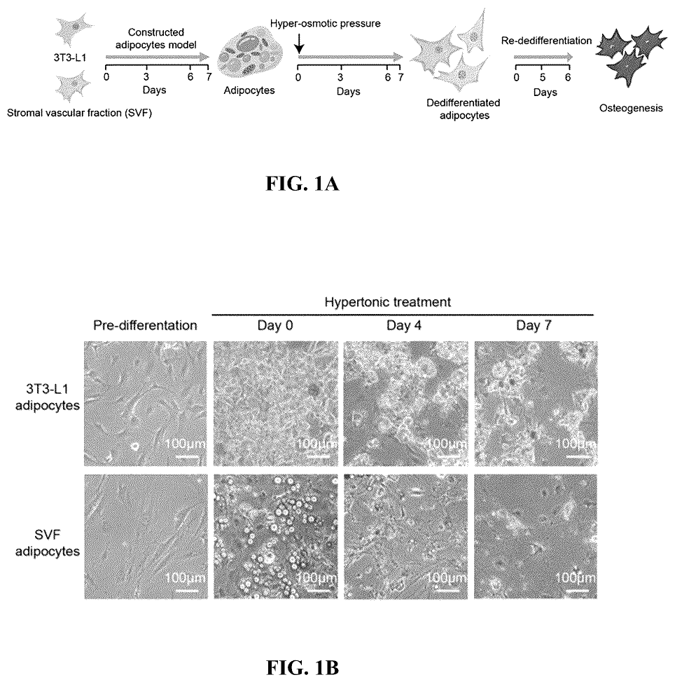

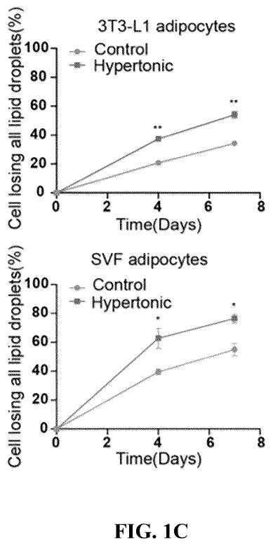

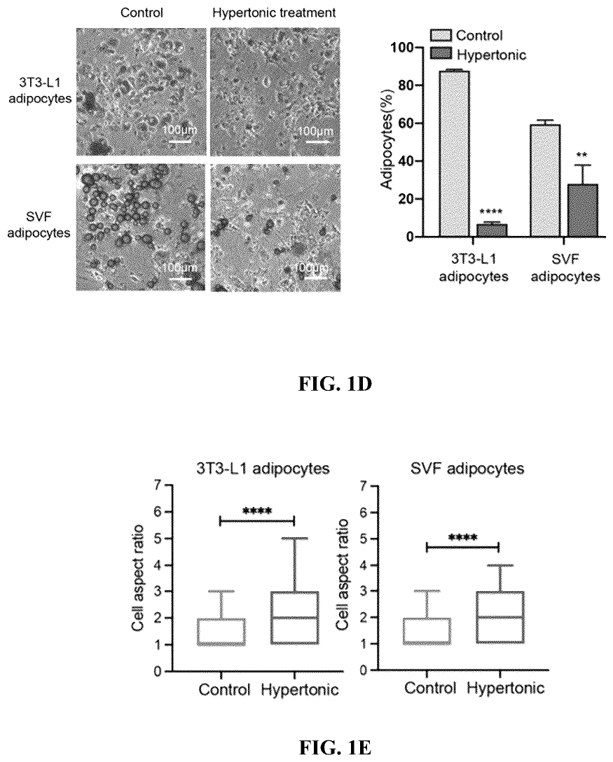

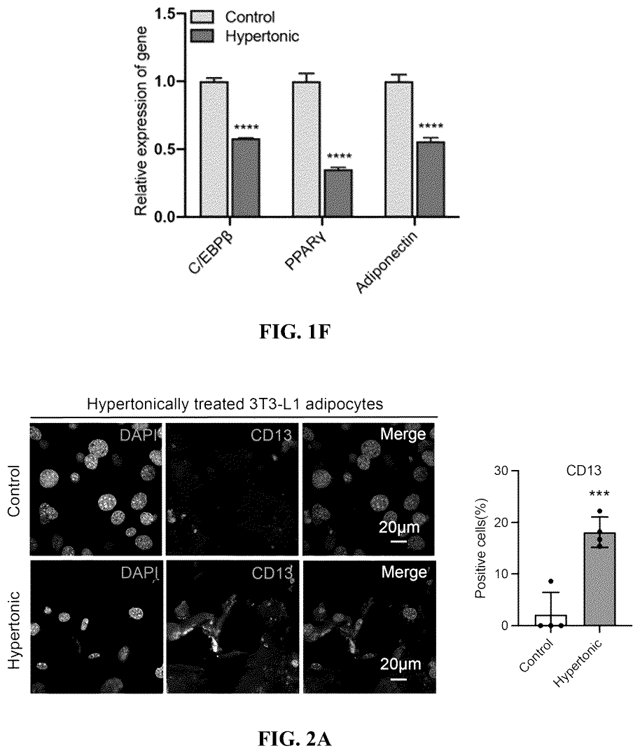

To address this, an examination was conducted to assess whether hypertonic treatment could induce dedifferentiation in the established 3T3-L1 adipocyte model. Turning to FIG. 1A, a diagram showing the experimental workflow was shown. Adipocytes derived from the stromal vascular fraction (SVF) were included as the positive control. Following the adipogenic culture of 3T3-L1 and SVF cells, the adipocytes were treated with the hypertonic medium for another 7 days, resulting in adipocytes with visible accumulation of lipid droplets and round morphology. The dedifferentiated cells were isolated for osteogenic differentiation.

The dedifferentiation process of the adipocytes was initiated by subjecting them to hypertonic treatment with 2% PEG-300 in the culture media, as previously described [11-12]. Over four to seven days of hypertonic treatment, approximately 40-70% of the 3T3-L1 and SVF adipocytes lost lipid droplets, which were significantly higher than the control isotonic treatments (FIGS. 1B-1C). Upon the completion of the 7-day treatment, Oil Red O staining was performed, confirming the reduction in lipid content and adipocyte count (FIG. 1D). Concurrently, a notable increase in the aspect ratios of cells in the hypertonic treatment group was observed (FIG. 1E), consistent with the elongated morphologies seen in previous studies on dedifferentiated adipocytes [2, 13]. That is, the dedifferentiated adipocytes will exhibit elongated morphologies.

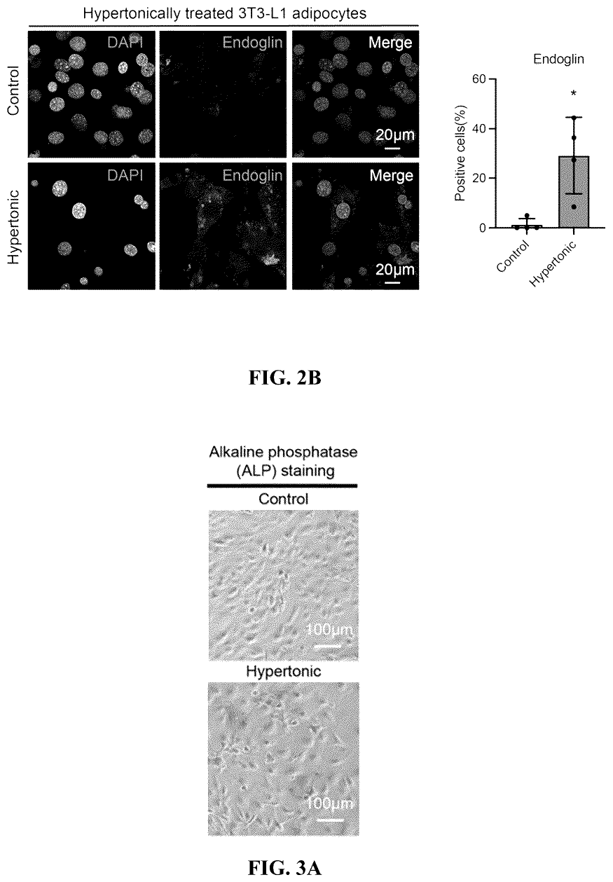

To confirm the dedifferentiation of 3T3-L1 adipocytes, an assessment of the expression of adipocyte marker genes was further conducted. Compared with the isotonic control, hypertonic treatment of 3T3-L1 adipocytes led to 40-60% reductions in the expression of adipogenic markers, including C/EBPβ, PPAR-γ, and adiponectin (FIG. 1F). Concurrently, immunostaining was employed to investigate CD13 and Endoglin, which are surface markers associated with dedifferentiated adipocytes. Quantitative analysis revealed significantly higher expression of CD13 and Endoglin in adipocytes of the hypertonic treatment than the isotonic control (FIGS. 2A-2B). Overall, these results indicated that hypertonic treatment could effectively induce dedifferentiation of 3T3-L1 adipocytes.

### Example 2

Re-Differentiation Capacity of Hypertonic-Treated 3t3-11 Adipocytes into Osteogenic and Chondrogenic Lineages

An important functional feature of the dedifferentiated adipocytes is the ability to re-differentiate into other mesenchymal lineages. Next, an evaluation was performed on the 3T3-L1 adipocytes to assess their capacity in osteogenic and chondrogenic re-differentiation after hypertonic treatment.

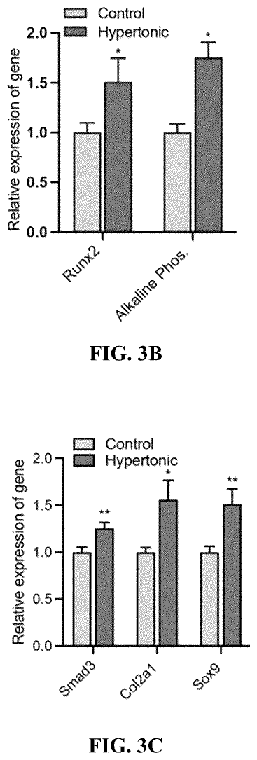

Osteogenic induction promoted the expression of alkaline phosphatase (ALP), a marker of the osteogenic lineage, in 3T3-L1 adipocytes receiving hypertonic treatment (FIGS. 3A-3C). This was accompanied by significantly increased expressions of Runx2, which is another osteogenic marker (FIG. 3B). In addition, chondrogenic induction of the 3T3-L1 adipocytes after hypertonic treatment led to an increased expression of chondrogenic marker genes, including Smad3, Col2a1 and Sox9 (FIG. 3C). Importantly, the control cells that received isotonic treatment did not display osteogenic nor chondrogenic differentiation capacity, in line with the adipogenic commitment of the 3T3-L1 cells. In summary, it can be concluded that hypertonic treatment effectively induces the multipotent dedifferentiation of 3T3-L1 adipocytes.

### Example 3

### MEVs Released by Adipocytes Under Hypertonic Conditions

Cellular transformation is commonly accompanied by autocrine signaling activities. Among them, extracellular vesicles (EVs) are a prominent mediator of cell-cell communication in many biological contexts including cancer. To examine the potential role of EV factors in regulating the hypertonic dedifferentiation of adipocytes, EVs were isolated from both isotonic and hypertonic treatments of 3T3-L1 adipocytes.

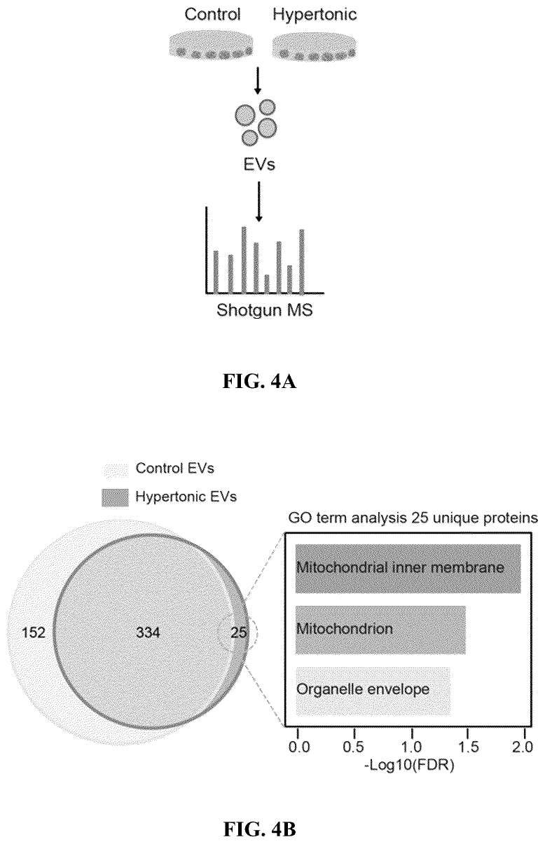

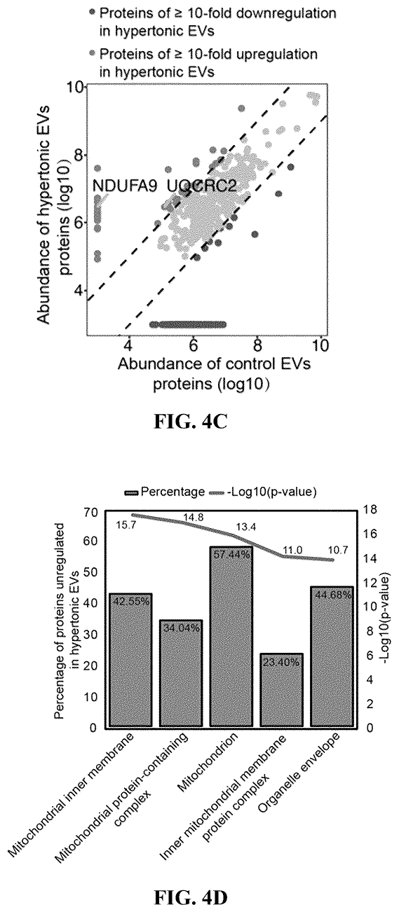

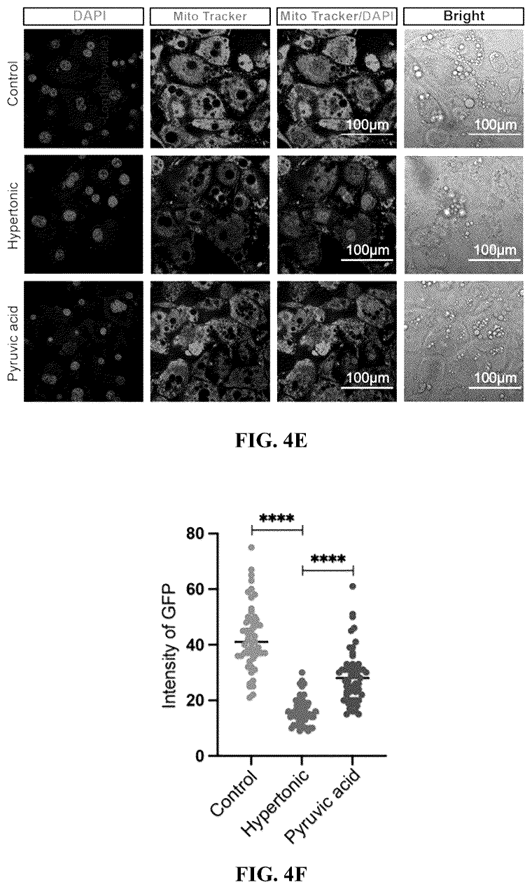

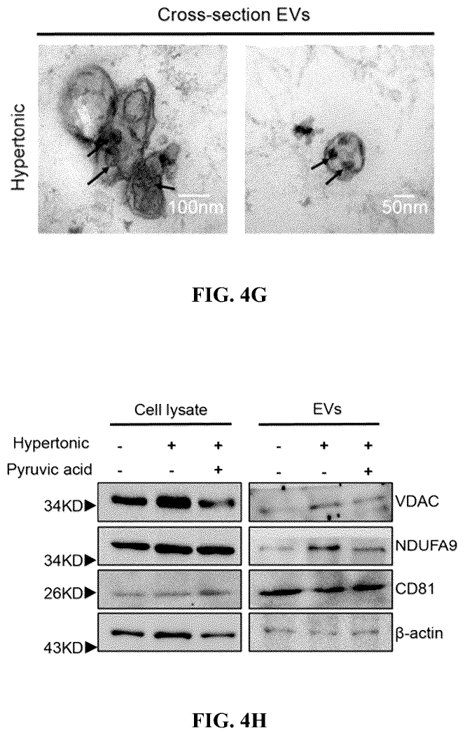

Referring to FIGS. 4A-4H, the hypertonic treatment prompted adipocytes to eject mitochondria via EVs. A comparison of their proteomic contents was conducted using LC-MS analysis (FIG. 4A). Overall, a total of 359 and 486 proteins (identified with ≥2 unique peptides) were detected in the EVs obtained from hypertonic- and isotonic-treated 3T3-L1 adipocytes, respectively. Among the two EV populations, 334 proteins were found to be common, while 25 proteins were uniquely identified in the hypertonic EVs (FIG. 4B). Intriguingly, Gene Ontology (GO) term annotation of the 25 hypertonic-unique proteins revealed enrichment of mitochondrial components (FIG. 4B and Table 1), which host the enzymatic complexes of oxidative phosphorylation and play a key role in ATP production and energy homeostasis of cells.

In addition, quantitative analysis identified 47 proteins with > tenfold upregulation in hypertonic EVs (FIG. 4C). Notably, GO term analysis ascribed more than 50% of the hypertonically up-regulated EV proteins as components of the mitochondrion (FIG. 4D), including NDUFA9 and UQCRC2, which are components of the NADH-ubiquinone oxidoreductase (complex I) and the ubiquinol-cytochrome c reductase (complex III) on the mitochondrial inner membrane, respectively. These results suggested that adipocytes would eject mitochondrial components through EVs in response to hypertonic treatment.

TABLE 1
The most represented GO terms of cellular components
of unique proteins from hypertonic EVs

| Gene Ontology | Cellular Component | | |
|---|---|---|---|
| No | GOID | Description | FDR |
| 1 | GO: 0005743 | Mitochondrial inner | 1.03E−02 |
| | | membrane | |
| 2 | GO: 0005739 | Mitochondrion | 3.16E−02 |
| 3 | GO: 0031967 | Organelle envelope | 4.29E−02 |

Hypertonicity is known to disrupt the ionic homeostasis of cells and reduce the electronic potential in mitochondria, consequentially inhibiting ATP production. Hence, an assessment of the status of mitochondria in adipocytes was conducted using MitoTracker Green (MTG), a fluorescent probe that accumulates in live-cell mitochondria in an electronic-potential-dependent manner. Compared to the isotonic control, hypertonic treatment markedly blocked MTG staining of mitochondria in adipocytes. Pyruvic acid is known to alleviate mitochondrial stress by improving electronic potential, and application of pyruvic acid (1 mM) to the hypertonic treatment effectively restored MTG staining (FIGS. 4E-4F). These results suggested that hypertonicity treatment could lead to disrupted electronic potential and mitochondrial stress in adipocytes.

Recent investigations have revealed that a spectrum of cells could eject damaged mitochondria from their cytosol [14-15]. Consequently, the hypothesis was formulated that adipocytes release mitochondria in EVs in response to the hypertonic stress on mitochondria.

To investigate this, electron microscopy (EM) was utilized to inspect the EVs released from 3T3-L1 adipocytes under hypertonic treatment. FIG. 4G revealed folded membranous structures within the EVs that resembled mitochondrial cristae. In parallel, hypertonic treatment also increased the levels of mitochondrial proteins VDAC and NDUFA9 in the EVs of 3T3-L1 adipocytes, which were reduced by pyruvic acid, alleviating mitochondrial stress (FIG. 4H). Simultaneously, no variations were observed in the levels of the pan-EV marker CD81, suggesting the specific effects on the mitochondrial components.

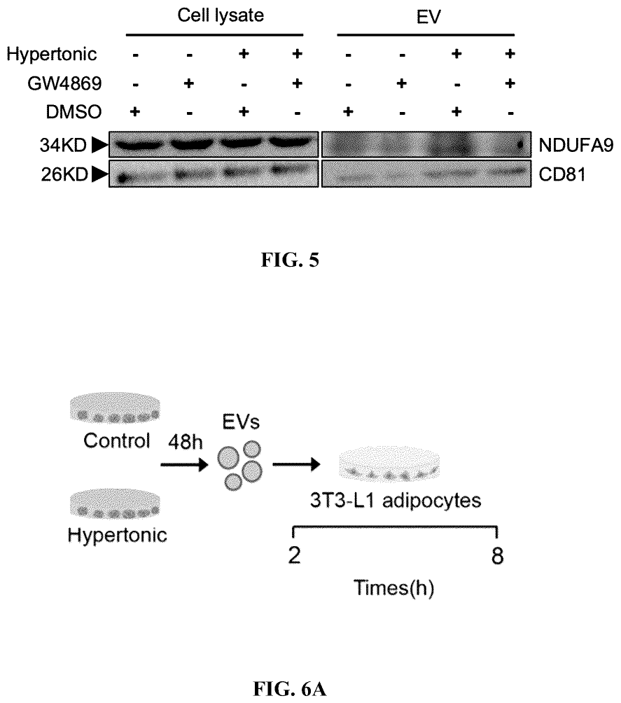

In addition, GW4869, a compound that blocks EVs secretion, substantially inhibited the EVs level of NDUFA9 induced by hypertonic treatments (FIG. 5). This supports that mitochondrial components were released through the EVs route during hyperosmotic stress. Taken together, these results suggest that hypertonic treatment leads to mitochondrial stress in adipocytes, resulting in ejection of mitochondrial components in EVs. These vesicles have been termed mitochondrial EVs (MEVs).

### Example 4

### Effects of Inflammatory Pathways in Promoting the Hypertonicity-Induced Adipocytes Dedifferentiation

The transfer of mitochondrial contents via extracellular vesicles (EVs) has been demonstrated to stimulate metabolic and inflammatory responses in recipient cells. Consequently, mitochondrial extracellular vesicles (MEVs) may serve as an autocrine factor governing adipocyte dedifferentiation.

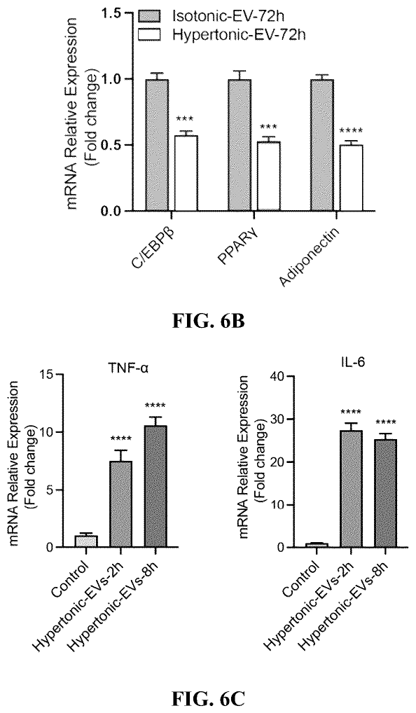

To validate this, MEVs were isolated from the hypertonic treatment of 3T3-L1 adipocytes and subsequently applied to naïve 3T3-L1 adipocytes cultured in isotonic conditions (FIG. 6A). The results showed that hypertonicity and mitochondrial EVs (MEVs) activates TNF-α signaling in 3T3-L1 adipocytes. After 72 hours, RT-qPCR analysis revealed a significant reduction of the expression of adipogenic markers, including C/EBPβ, PPAR-γ, and adiponectin (FIG. 6B). These results suggested a potential role of MEVs as the mediator of hypertonic-induced adipocyte dedifferentiation.

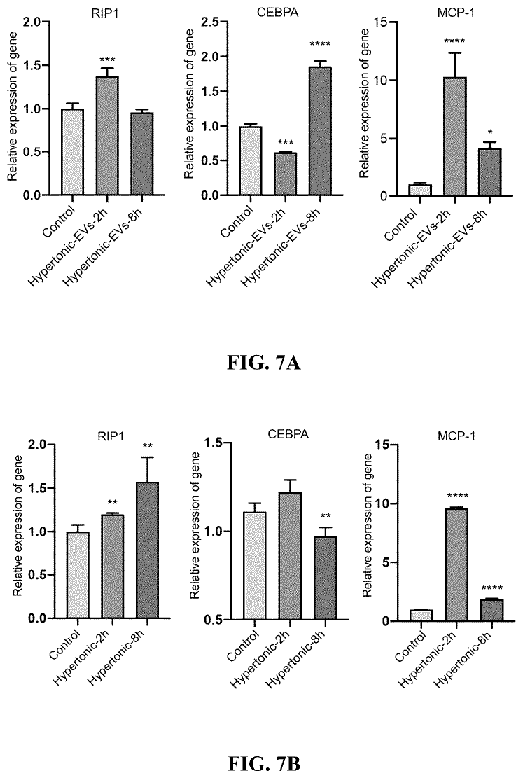

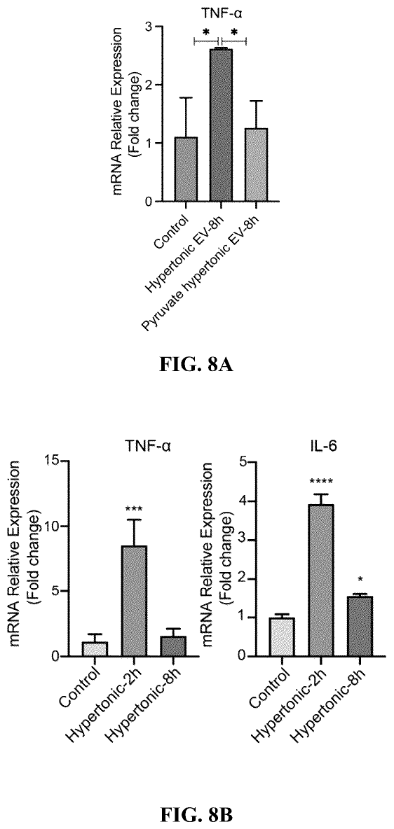

Extracellular mitochondria have been demonstrated to promote inflammatory processes. The subsequent investigation aimed to determine whether hypertonic MEVs could influence inflammatory pathways in adipocytes. Indeed, application of hypertonic MEVs to 3T3-L1 adipocytes significantly induced the expression of a series of inflammatory genes, including TNF-α, IL-6, RIP1, CEBPA, and MCP-1 (FIG. 6C and FIG. 7A). In particular, a robust 4- and 8-fold induction was observed for the key inflammatory genes TNF-α and TL-6 after 2-h MEVs treatment, respectively. The induction of both genes remained high at 8 hours post treatment, suggesting the long-lasting effect of hypertonic MEVs. Importantly, reducing mitochondria stress by pyruvic acid prevented the hypertonic EVs from inducing TNF-α expression (FIG. 8A). Given that pyruvic acid decreases the mitochondrial contents in EVs, these results suggested that hypertonic MEVs could stimulate inflammatory signaling in adipocytes. Subsequently, the inflammatory responses of adipocytes in the hypertonic culture were investigated. Similar to the hypertonic EVs treatment, 2 hours of hypertonic culture induced the expression of inflammatory genes TNF-α, IL-6, RIP1, CEBPA, and MCP-1 in 3T3-L1 adipocytes (FIG. 8B and FIG. 7B).

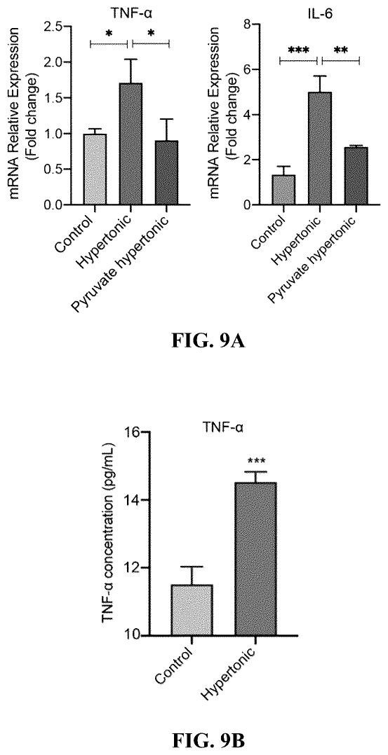

Interestingly, the expression levels of TNF-α, IL-6, CEBPA, and MCP-1 declined substantially after 8 hours of hypertonic culture. This was in contrast to the long-lasting effect of hypertonic EVs and could be due to adaptation of adipocytes to the relatively mild hypertonicity. Notably, addition of pyruvic acid to the hypertonic culture abolished the induction of TNF-α and IL-6 expression (FIG. 9A), further supporting the role of mitochondrial stress in the effects of hypertonicity. Importantly, ELISA assay revealed an increased level of TNF-α protein in the media of hypertonic-treated adipocytes (FIG. 9B). Altogether, these results indicated that hypertonic stress and mitochondrial EVs could activate inflammatory signaling in adipocytes.

### Example 5

### The Role of TNF-α and Wnt/β-Catenin Signaling in Hypertonicity-Induced Adipocyte Dedifferentiation

Previous studies have highlighted the anti-adipogenic activity of the TNF-α inflammatory signaling [8, 16]. In order to investigate the role of TNF-α in hypertonicity-induced adipocyte dedifferentiation, TNF-α neutralizing antibody (20 ng/ml) was administered to 3T3-L1 adipocytes in the hypertonic culture.

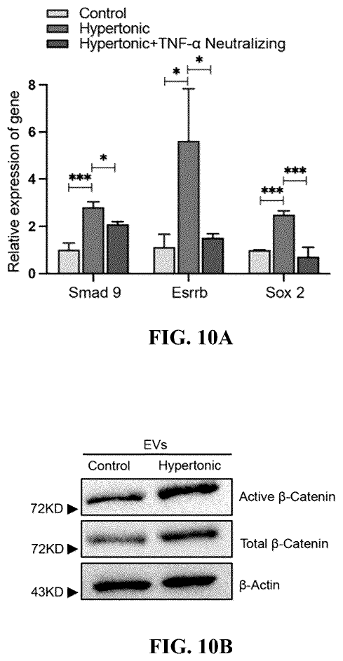

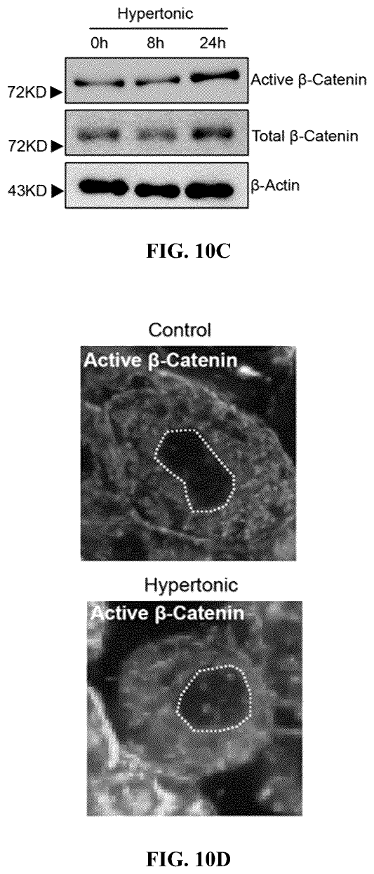

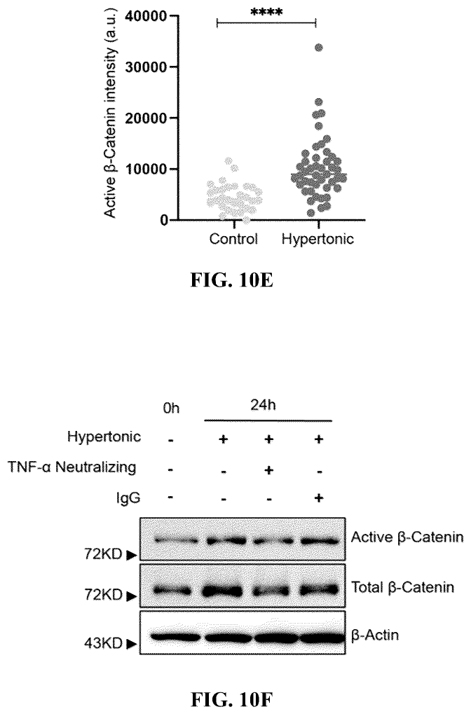

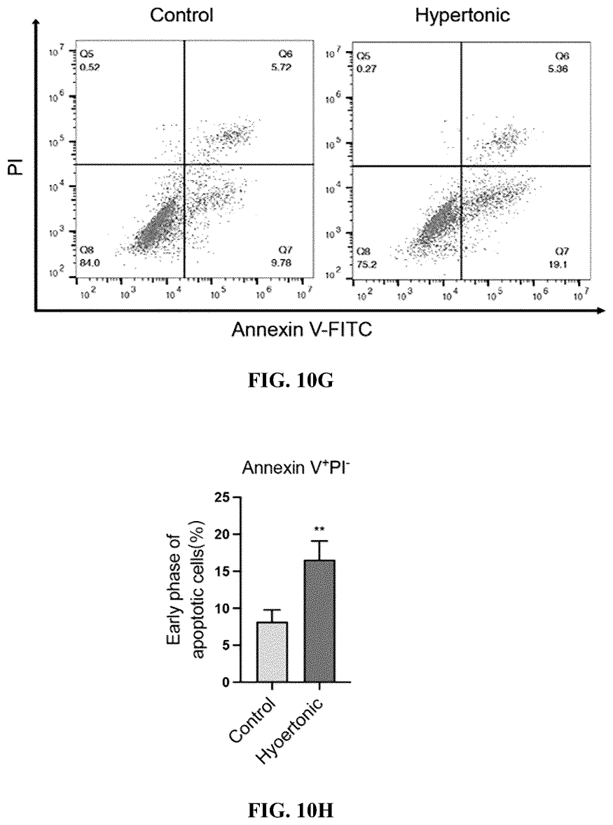

FIGS. 10A-10H showed that TNF-α activates β-catenin and apoptosis in adipocytes under hypertonic treatment. Compared to the control antibody, TNF-α neutralizing antibody significantly inhibited the hypertonicity-induced expression of stem cell genes, including Esrrb, and Sox2. Though not significant, a modest reduction of Smad 9 was also observed after neutralizing TNF-α in the dedifferentiating adipocytes (FIG. 10A). These results pointed to a key role of TNF-α in hypertonicity-induced adipocyte dedifferentiation.

The Wnt/β-catenin pathway is also known to inhibit adipogenesis and promote the dedifferentiation of mature adipocytes. In fact, activation of β-catenin was reported to underlie hypertonic dedifferentiation of adipocytes [11]. Subsequently, an assessment was conducted to determine the impact of hypertonic EVs treatment on β-catenin in adipocytes.

Western blot demonstrated stabilization of β-catenin and accumulation of non-phosphorylated (active) β-catenin in 3T3-L1 adipocytes after 24 hours of hypertonic MEVs treatment (FIG. 10B). This was also observed in 3T3-L1 adipocytes after 24 hours of hypertonic culture (FIG. 10C). In parallel, immunostaining revealed that the nuclear accumulation of active β-catenin in hypertonic culture (FIGS. 10D-10E). Therefore, in addition to inducing TNF-α, hypertonic stress and the mitochondrial EVs also activate the anti-adipogenic Wnt/β-catenin signaling.

TNF-α has been reported to induce the stabilization of β-catenin and inhibit adipogenesis [8]. Hence, the significance of TNF-α in the hypertonic activation of β-catenin during adipocyte dedifferentiation was evaluated. Turning to FIG. 10F, the application of the TNF-α neutralizing antibody markedly blocks the stabilization of total β-catenin and non-phosphorylated (active) β-catenin in hypertonic culture. These findings illustrate the essential role of TNF-α in the hypertonic activation of Wnt/β-catenin signaling, which orchestrates adipocyte dedifferentiation.

### Example 6

Hypertonicity-Induced Adipocyte Dedifferentiation and Apoptosis: Assessment and Therapeutic Intervention with BML-284

The hypertonic dedifferentiation of adipocytes typically requires up to 7 days of culture in 2% PEG-300. Considering that TNF-α is recognized as a proinflammatory cytokine capable of inducing adipocyte apoptosis, an evaluation of the apoptotic status of adipocytes was carried out using AnnexinV-FITC/PI staining. Referring to FIGS. 10G-10H, a substantial increase in cells in the early stages of apoptosis, as indicated by Annexin V staining, was observed compared to the isotonic control. Simultaneously, no difference was observed in PI-positive cells in the early phase of apoptosis, which could potentially be attributed to the loss of such cells during the washing process prior to harvesting. It was concluded that the extended hypertonic treatment could lead to apoptosis alongside the dedifferentiation of adipocytes.

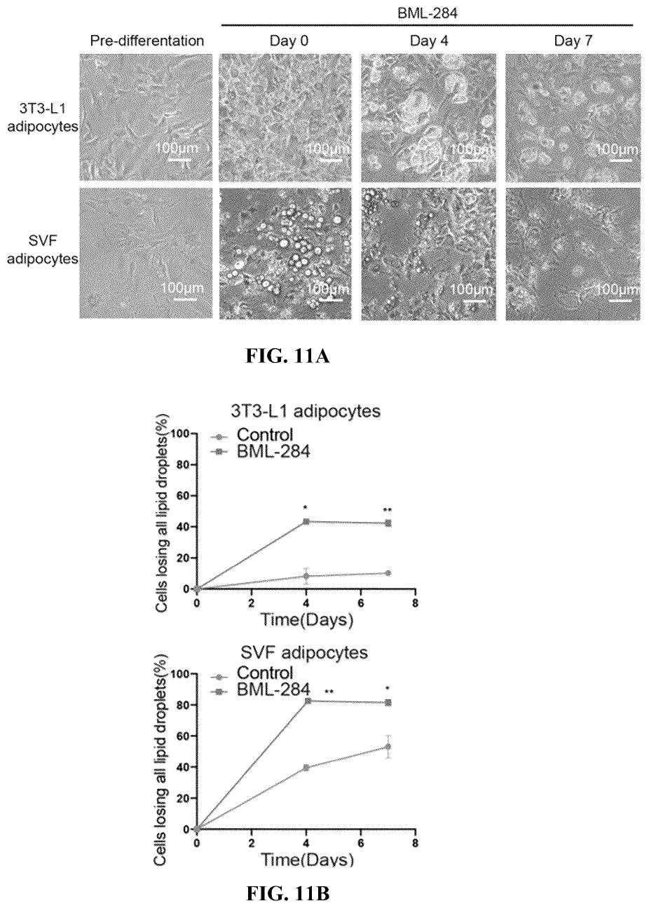

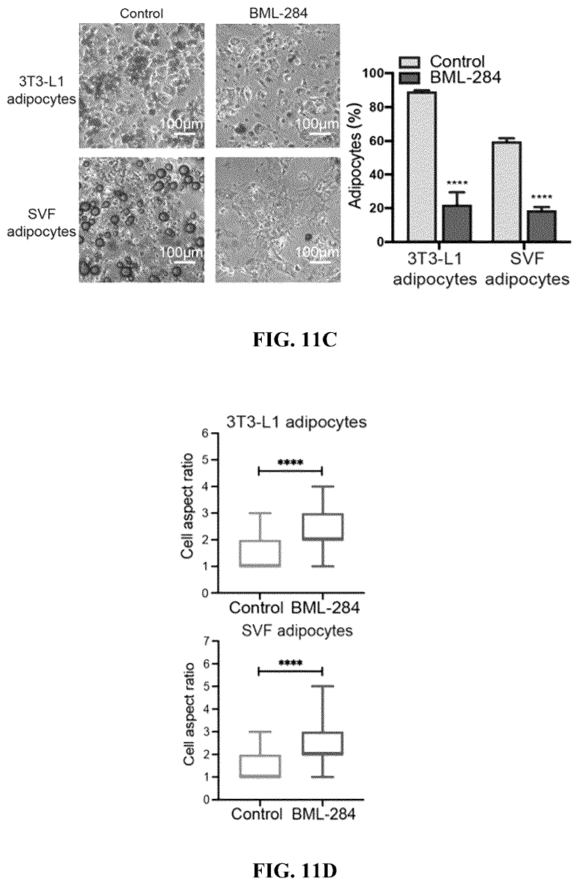

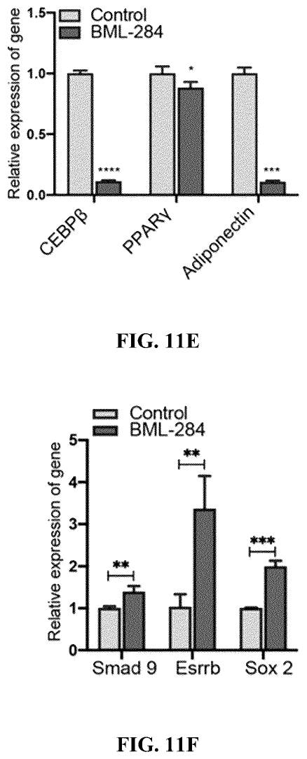

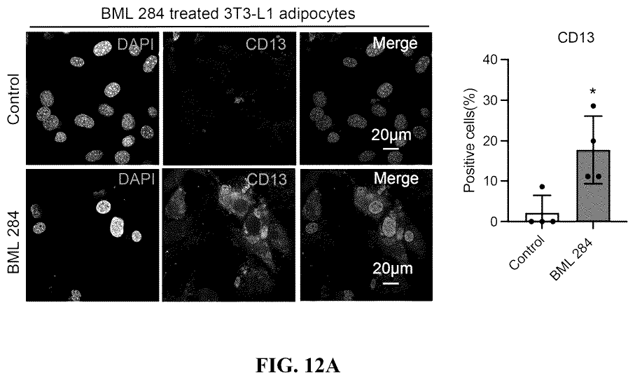

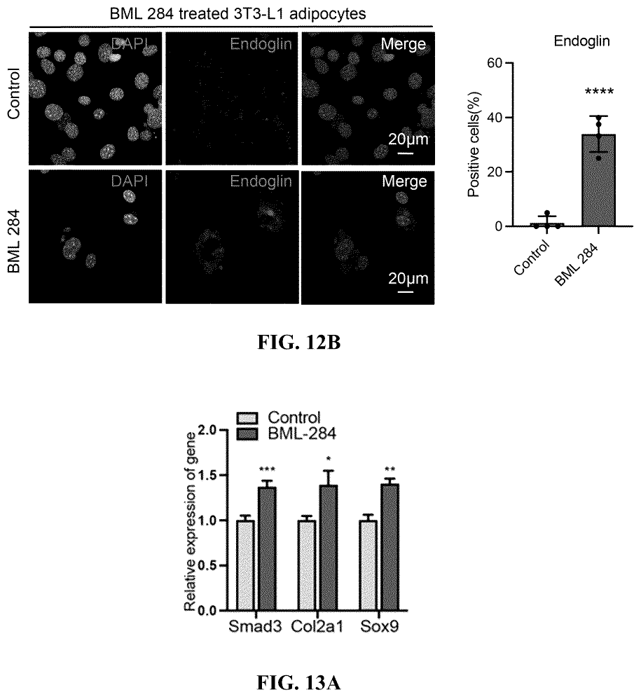

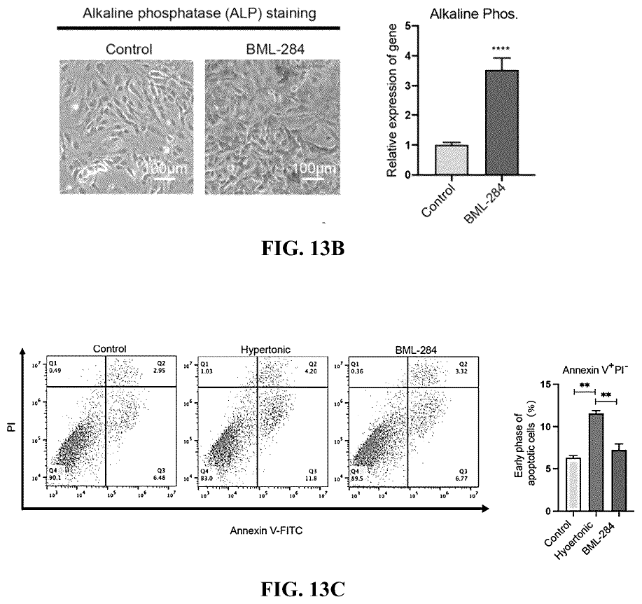

To circumvent the apoptotic effects of hypertonicity and TNF-α in inducing adipocyte dedifferentiation, BML-284, a small compound that stabilizes β-catenin and directly activates the Wnt/β-catenin signaling, was utilized. 3T3-L1 and SVF adipocytes in isotonic cultures were subjected to treatment with BML-284 (10 mM), and subsequent assessments were made regarding lipid droplets and morphological changes. Strikingly, After 4 days of BML-284 treatment, a marked reduction in visible lipid droplets was observed (FIGS. 11A-11B). In 3T3-L1 adipocytes, the loss of lipid droplets amounted to 40%, while in SVF adipocytes, it reached 80%. This is accompanied by significant increases of cells with elongated morphology in BML-284-treated 3T3-L1 and SVF adipocytes (FIG. 11C). These results indicated the anti-adipogenic activity of BML-284, which was further supported by decreased Oil red staining (FIGS. 11C-11D). Simultaneously, there were significant reductions in the expression of adipogenic genes (C/EBPβ, PPAR-γ, and adiponectin), coupled with significant increases in the expression of marker genes (Smad 9, Esrrb, and Sox2) and proteins (CD13 and endoglin) of multipotency in BML-284 treated cells (FIGS. 11E-11F and FIG. 12A-12B). Importantly, the re-differentiation assays revealed increased expression of osteogenic and chondrogenic markers in BML-284-treated adipocytes after inducing treatments correspondingly (FIGS. 13A-13B). Altogether, these results indicated that direct activation of β-catenin signaling by BML-284 could induce adipocyte dedifferentiation.

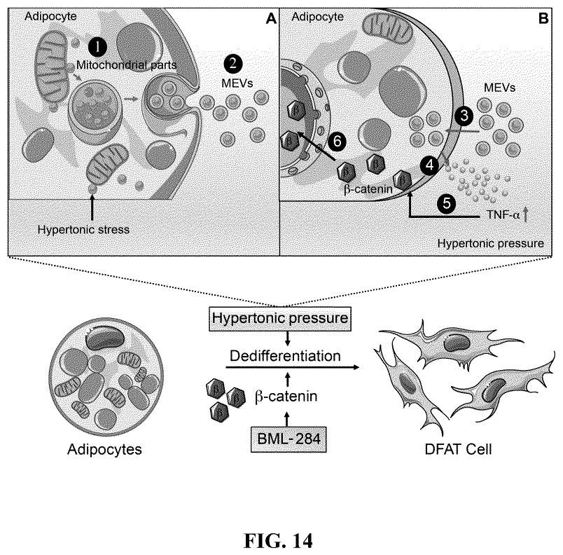

The subsequent step involved a comparison of the effects of BML-284 and the hypertonic treatment on cell apoptosis. Notably, while the hypertonic treatment induced a significantly level of apoptosis in 3T3-L1 adipocytes, BML-284 did not lead to significant apoptosis (FIG. 13C). Overall, these results demonstrated that BML-284 treatment could effectively induce adipocyte dedifferentiation while circumventing the apoptotic effect of hypertonic treatment. Elucidating the mechanisms that dictate the apoptosis/dedifferentiation fate would pave the way for effectively harnessing adipocytes for regenerative medicine (FIG. 14).

Osmotic stress can take place in a diverse array of conditions with physiologic or pathologic influences on the adipocytes. For example, hyperosmotic stress of adipocytes was reported to inhibit insulin signaling and induce insulin-resistance. Zhu et al. reported that the adipocytes in breast cancer tissues could undergo the adipocyte mesenchymal transition (AMT) that generate multiple cell types constituting an inflammatory and tumor-promoting stroma [17]. Although the mechanism of AMT remains unclear, it is important to note that the osmotic stress and compression in breast cancer have been reported to activate β-catenin signaling and induce the adipocytes dedifferentiation with multi-lineage redifferentiation potentials [11]. The results suggest that hypertonic MEVs mediate β-catenin activation and adipocyte dedifferentiation in response to osmotic stress. It would be of interest to investigate whether hypertonic stress of mitochondria and MEVs of adipocytes are implicated in the malignant AMT of breast adipose tissue in vivo. This could provide a potential therapeutic target in the breast cancer microenvironment.

In addition, recent studies have demonstrated that various cells could eject mitochondria to regulate homeostasis. For example, Nicolás-Ávila et al. found that cardiomyocytes release damaged mitochondria in extracellular vesicles (EVs) to maintain proper heart function [18]. It is reported here that hypertonic treatment of adipocytes can result in EV-mediated mitochondrial ejection. Mechanistically, the fluctuation of the cellular osmolarity could perturb the ion homeostasis and electronic potential in mitochondria, leading to damages of the components. This is supported by the data that pyruvate could reduce the hypertonic MEVs. Similarly, Rosina et al. have reported that thermogenically stressed brown adipocytes eject dysfunctional mitochondrial parts in EVs to prevent failure of the thermogenic program [14]. It remains unclear about the cellular process and mechanisms of releasing mitochondrial components via EVs. It would be interesting to compare MEVs from different conditions for their components and activities in inducing adipocyte dedifferentiation. Accumulating evidence also indicate that adipocyte dedifferentiation plays an important role in tissue homeostasis. Zhang et al. reported that dermal adipose tissue undergoes dedifferentiation and redifferentiation in hair cycle and wound healing[19]. In line with this, lineage tracing and single-cell RNA sequencing have revealed myofibroblasts derived from adipocytes during wound healing [20]. It is worth noting that wound healing is associated with the activation of inflammatory cytokines, including TNF-α.

In the studies of both Nicolás-Ávila et al. and Rosina et al., immune cells such as macrophages play a critical role in removing the ejected mitochondria and maintaining the homeostasis of the source tissue. Therefore, how MEVs function with the inflammation/immune system to induce adipocyte dedifferentiation in vivo would need further investigation.

### Example 7

### Materials and Methods

### Cell Culture

3T3-L1 (ATCC CL-173) cells were cultured in DMEM medium (Thermo Fisher, product no. 11965126) containing 10% FBS, 2 mM L-glutamine and 100 mg/ml of penicillin-streptomycin (Thermo Fisher).

C57BL/6J mice were used for the present invention and maintained in a 12:12 h light-dark cycle. All mouse experiments were performed in accordance with the protocol approved by the Institutional Animal Research Ethics Sub-Committee of City University of Hong Kong and Department of Health, The Government of The Hong Kong Special Administrative Region. 6 to 8-week-old female were anesthetized with 4% of isoflurane by inhalation, and then inguinal, retroperitoneal WAT were removed. The tissues were shredded after washing in sterile PBS. Afterwards, the tissue pieces were incubated in type II collagenase (Sigma, product no. C6885) for 1 h at 37° C. before passed through a sterile 100 μm filter. SVF was obtained after 5 min centrifugation at 1000 rpm. Culture medium (DMEM supplemented with 10% FBS and 1% penicillin-streptomycin (all obtained from Thermo Fisher) was added to resuspend the cells, followed by culturing in 100 mm culture dishes. The following day, the culture medium was changed to remove the dead and non-adherent cells.

### 3T3-L1 and SVF Differentiation

1.25×10⁵ 3T3-L1 were seeded in 6-well dishes and cultured at 37° C. and 5% CO₂. The 10 μg/ml insulin (MedChemExpress, product no. HY-P0035), 1 μM dexamethasone (MedChemExpress, product no. HY-14648), 0.5 mM 3-isobutyl-1-methylxanthine (IBMX) (MedChemExpress, product no. HY-12318) and 2 μM rosiglitazone (Sigma, product no. R2408-10MG) (Differentiation medium) were supplemented in culture medium to constitute the differentiation medium for 2 days. Differentiation to SVF was induced by differentiation medium. The SVF cells were incubated for 5 days with differentiation medium. The medium was then changed to DMEM containing 10% FBS, 1×P/S, and 10 μg/ml insulin for 48 h, followed by changing to the culture medium.

### Oil Red O Staining

Adipocytes were stained with Oil red O staining kit (Solarbio, product no. G1262) solution to detect the lipid droplets according to manufacturer's instructions. Specifically, cells were fixed with 10% formalin for 20 min at room temperature, followed by washing with 60% isopropanol for 5 min. Subsequently, staining was conducted with ORO solution for 20 min at room temperature to enable binding of the dye to the lipid droplets. Finally, the cells were washed with distilled water to eliminate any residual dye.

### Hypertonic-Induce Dedifferentiation of Adipocytes

To induce hypertonic shock, the adipocytes (3T3-L1 or SVF) were treated with a medium containing 2% polyethylene glycol 300 (PEG 300) diluted in culture medium. Specifically, the adipocytes were exposed to the hypertonic deditfferentiation medium, composed of DMEM supplemented with 10% FBS and 1% penicillin-streptomycin (all obtained from Thermo Fisher) and 2% PEG-300 (MedChemExpress, product no. HY-Y0873), and maintained at 37° C. and 5% CO₂. The medium was changed every 3 days, being careful to avoid shaking the dish to allow the hypertonic medium to diffuse slowly in the culture dish.

### Osteogenic Differentiation

The osteogenic and chondrogenic re-differentiation capacities of dedifferentiated adipocytes were also examined. To induce osteogenic differentiation, 1×10⁶ cells were seeded in 6-well plates and incubated overnight at 37° C. and 5% CO₂. The culture medium was then replaced with StemPro Osteogenesis Differentiation medium (Thermo Fisher, product no. A1007201) and cells were maintained in this medium for up to 14 days, with medium renewal every 3 days. This medium contains specific factors that promote differentiation towards the osteogenic lineage, allowing the cells to deposit mineralized extracellular matrix and express osteogenic markers.

### Alkaline Phosphatase Assay

Alkaline phosphatase (ALP) staining was conducted on Day 9 of the feeder cell-mediated reprogramming process, following the manufacturer's instructions for the Alkaline Phosphatase Detection Kit (Sigma, product no. SCR004). The staining was performed to detect the activity of ALP, a marker of pluripotency, and to assess the success of the reprogramming process.

### MitoTracker Staining

To stain cells with MitoTracker (CST, product no. FM 9074), the 1 mM stock solution should be diluted directly into normal growth media to achieve a staining concentration ranging from 400 nM. The live cells were incubated for 15 minutes at 37° C. Following incubation, live imaging is necessary to visualize the staining pattern within the cells.

qPCR Gene Expression Analysis

Total RNA was isolated using the RNA Extraction Kit (Takara, product no. 9767) in accordance with the manufacturer's recommended protocol. The first-strand cDNA was synthesized using the PrimeScript RT Reagent Kit (Takara, product no. RR047A) for RT-PCR. In brief, a 10 μl system containing 2 μl of 5× PrimeScript RT Master Mix (Perfect Real Time) and RNase-Free Distilled Water and RNA solution, with a total RNA amount of 500 ng, was transferred to the ABI ProFlex PCR System (2×96-well) to perform reverse transcription. RT-PCR was then conducted using the Ex-Taq PCR kit (Takara, product no. RR820A) following the manufacturer's instructions. All RT-qPCR primer sequences were included in Table 2.

TABLE 2
primer sequences used for qPCR validation

| Primers | | |
|---|---|---|
| qRT-PCR | Forward Primer 5′-3′ | Reverse primer 5′-3′ |
| 18SRNA | CTGAGAAACGGCTACCACATC | GCCTCGAAAGAGTCCTGTATTG |
| | (SEQ ID NO: 1) | (SEQ ID NO: 2) |
| TNF-α | ATGGCCTCCCTCTCATCAGT | CTTGGTGGTTTGCTACGACG |
| | (SEQ ID NO: 3) | (SEQ ID NO: 4) |
| RIP1 | CACTGCGATCATTCTCGTCCTG | GACTGTGTACCCTTACCTCCGA |
| | (SEQ ID NO: 5) | (SEQ ID NO: 6) |
| IL-6 | GTTCTCTGGGAAATCGTGGA | GCATTGGAAATTGGGGTAGG |
| | (SEQ ID NO: 7) | (SEQ ID NO: 8) |
| MCP-1 | AGCCAACTCTCACTGAAGCC | GGACCCATTCCTTCTTGGGG |
| | (SEQ ID NO: 9) | (SEQ ID NO: 10) |
| Col2a1 | CCGCAGTCACTCCAGGAT | TGCAGTCTGCCCAGTTCA |
| | (SEQ ID NO: 11) | (SEQ ID NO: 12) |
| Sox9 | AGCTCACCAGACCCTGAGAA | TCCCAGCAATCGTTACCTTC |
| | (SEQ ID NO: 13) | (SEQ ID NO: 14) |
| Smad3 | GTCAACAAGTGGTGGCGTGTG | GCAGCAAAGGCTTCTGGGATAA |
| | (SEQ ID NO: 15) | (SEQ ID NO: 16) |
| Esrrb | GTGCGCAGGTACAAGAAACT | CCAGGTTCTCAATGTACATCGA |
| | (SEQ ID NO: 17) | (SEQ ID NO: 18) |
| Sox2 | GCGGAGTGGAAACTTTTGTCC | GGGAAGCGTGTACTTATCCTTCT |
| | (SEQ ID NO: 19) | (SEQ ID NO: 20) |
| Smad9 | CGGATGAGCTTTGTGAAGG | GGGTGCTCGTGACATCCT |
| | (SEQ ID NO: 21) | (SEQ ID NO: 22) |
| Runc2 | GCCGGGAATGATGAGAACTA | GGTGAAACTCTTGCCTCGTC |
| | (SEQ ID NO: 23) | (SEQ ID NO: 24) |
| ALP | TGAGCGACACGGACAAGA | GGCCTGGTAGTTGTTGTGAG |
| | (SEQ ID NO: 25) | (SEQ ID NO: 26) |

### Immuno/Fluorescent Labeling and Microscopy

Twelve hours before the experiment, cells were seeded onto cover slips in 12-well dishes and cultured overnight at 37° C. and 500 CO₂. Cells were fixed with 200 paraformaldehyde/PBS for 10 min at 4° C. Next, the cells were either directly incubated with 1% BSA/PBS for 1 h or first permeabilized with 0.1% Triton X-100/PBS for 10 min at 4° C. before blocking with 1% BSA/PBS. Subsequently, CD13 primary antibody (Santa Cruz, product no. sc-13536 1:100) or Endoglin primary antibody (Santa Cruz, product no. sc-18838 1:100) was incubated with cells overnight at 4° C. Following 3 times washing in 0.5% tween/PBS, cells were incubated with secondary antibody (CST, product no. 8890S) in 1% BSA/PBS (1:400) in darkness for 1.5 hours at room temperature. After further washing, cells were mounted with DAPI (Thermo Fisher, product no. 62247) and examined with a Nikon A1HD25 confocal microscope.

### Western Blotting

To obtain lysates, cells were washed twice with cold PBS and lysed with 1 mL of RIPA buffer (Beyotime, product no. P0013B) at 4° C. for 1 hour with gentle rotation. The lysate was subsequently centrifuged at 12,000 g for 20 min at 4° C., and the resulting supernatant was quantified the protein concentration. Protein extracts were then separated by SDS-PAGE and transferred onto a NC membrane (Bio-Rad, product no. 1620112). The membranes were incubated overnight at 4° C. with antibodies against VDAC (CST, product no. 4866s, 1:1000), NDUFA9 (Abcam, product no. ab14713, 1:1000), CD81(Santa Cruz, product no. sc-166029, 1:1000), Non-phospho (Active) β-Catenin (CST, product no. 8814, 1:2000), β-Catenin (CST, product no. 9562, 1:1000) and 3-actin (Santa Cruz, product no. sc-47778, 1:1000). Following incubation with the appropriate secondary antibody (Abcam, product no. ab205719; Thermo Fisher, product no. 31460, 1:4000), the protein bands were visualized using an enhanced chemiluminescence (ECL) kit (Bio-Rad, product no. 170-5061) and captured using a Bio-red ChemiDoc.

### Cellular Drug Treatment

To inhibit EV secretion, adipocytes were treated with 10 μM GW4869 (Sigma-Aldrich, product no. D1692) in isotonic and hypertonic cultures. An equivalent volume of DMSO was added to the control samples. After two days, the supernatants were collected for EV isolation and analysis.

To mitigate mitochondrial stress, adipocytes were treated with 1 mM pyruvic acid (Sigma-Aldrich, product no. 107360) in hypertonic cultures. An equivalent volume of DMSO was added to the control samples. Following the treatment, the cells were either subjected to MTG staining or the supernatants were collected for EV isolation and analysis.

### EVs Isolation and Protein Digestion

The 3T3-L1 adipocytes were treated by isotonic and hypertonic medium. After 48 h culture, conditional mediums were collected. The EVs were isolated by a sequential ultracentrifugation includes removing supernatant by 300 g for 10 min, 2000 g for 15 min, 10000 g for 70 min and collecting the sediment by 120000 g for 2 h. The pellets were washed once with PBS and precipitated at 120000 g for 70 min. Purified EVs pellets were vacuum dried and then were resuspended in 100 μL of buffer containing 8 M urea (Sigma, product no. U1250) and 50 mM NH₄HCO₃ (Alfa Aesar, product no. 14249). The resuspended suspensions contain approximately 80 μg protein each as determined by DC protein assay (Bio-Rad, product no. 5000114). In-solution digestion was performed for the following LC-MS/MS analysis. Briefly, the EVs protein suspensions were reduced with 20 mM DTT for 10 min at 95° C., followed by alkylation with 50 mM IAA for 30 min at RT in dark. The solutions were then diluted with 50 mM NH₄HCO₃ buffer (pH 8.0) to make the urea concentration lower than 1 M. MS-grade trypsin (Thermo Fisher, product no. 90058) coupled with Lyc-C protease was added into the solution (pH 8.0) at an enzyme/protein ratio of 1:25 to digest the protein at 37° C. overnight. The resulting peptide samples were desalted using C18 tips (Thermo Fisher, product no. 87784) and resuspended with 0.1% formic acid buffer (Thermo Fisher, product no. 85178) for LC-MS/MS analysis.

### LC-MS/MS Analysis

The peptide samples were analyzed using EASY-nLC 1200 system combined with a Q Exactive HF mass spectrometry (Thermo Scientific). For each injection, 6 L loading sample containing around 0.5 μg peptide was separated by a C18 nano-column (250 nm, 75 μm, 3 μm, PepSep, Denmark) (Thermo Fisher, product no. 87784) at a flow rate of 250 nl/min. The 75 min reversed-phase gradient was achieved by mobile phase A (0.1% formic acid in ultrapure water) and mobile phase B (0.1% formic acid/80% acetonitrile in ultrapure water). MS recording was operated in the range of 350 to 1800 m/z with a mass resolution of 120000. The positive ion mode was employed with the spray voltage at 2000V and a spray temperature of 320° C. The resolution of dd-MS2 was 30000 with a 1×10⁵ of AGC target. The Maximum IT was set at 60 ms, and the loop count was 12. The isolation window was 1.6 m/z, and the fixed first mass was 120.0 m/z.

### Database Search

Raw files created by XCalibur 4.0.27 (Thermo Fisher) software were analyzed using Proteome Discoverer software (version 2.2, Thermo Fisher) against the UniProt mouse protein database in Sequest HT node. The precursor and fragment mass tolerances were set to 10 ppm and 0.02 Da, respectively. A maximum of two missed cleavage sites of trypsin was allowed. Carbamidomethylation (C) was set as static modification, and oxidation (M) and acetyl (protein N terminal) were set as variable modifications. The false-discovery rates (FDRs) of peptide spectrum matches (PSMs) and peptide identification were determined using the Percolator algorithm at 1% based on q value. For label-free quantification, the Minora Feature Detector node was used in the processing workflow, and the Precursor Ions Quantifier node and the Feature Mapper node in the consensus workflow. Normalization of the quantitative values was performed in Proteome Discoverer, based on the total peptide intensity of the samples.

### Transmission Electron Microscopy Analysis of Extracellular Vesicle

EVs were fixed with a solution of 5% formaldehyde and 2% glutaraldehyde in 0.1M cacodylate buffer (pH 7.2) overnight at 4° C. After washing with 0.1M cacodylate buffer, the samples were further fixed with a solution of 2% osmium tetroxide in 0.1M cacodylate buffer for 2 hours at room temperature, in the dark. Following another wash with 0.1M cacodylate buffer, the samples were dehydrated using a series of ethanol solutions with increasing concentrations (30%, 50%, 70%, 80%, 90%, 95%, and 100%) and further dehydrated with 70% and 100% acetone. The samples were then infiltrated with Spurr's resin at increasing concentrations of 25%, 50%, 75%, and 100%. After polymerization for 2 days at 70° C., ultrathin sections were prepared using a diamond knife, collected onto butvar-coated 100 mesh grids (EMS, product no. FF100-CU), and counterstained with UranyLess (EMS, product no. 22409) for 10 min and lead citrate for 1 min. Finally, the samples were imaged using a transmission electron microscope (Philips Technai 12) to visualize the internal structure and morphology of the EVs.

### INDUSTRIAL APPLICABILITY

Multipotent dedifferentiation of adipocytes holds strong promises to revolutionize regenerative medicine. However, the application potential is hampered by the unclear mechanisms and low efficiency. MEVs serve as an early warning signal and plays a protective role in various physiological and pathological processes. The produced MEVs can serve as biomarkers for disease diagnosis and monitoring, providing valuable insights into cellular and tissue conditions. Therefore, the present invention has therapeutic applications in regenerative medicine and tissue engineering, facilitating processes such as cell proliferation, angiogenesis, and extracellular matrix remodeling.

### Abbreviations

SVF: Stromal vascular fraction
EVs: Extracellular vesicles
MEVs: Mitochondrial extracellular vesicles
DFAT.: Dedifferentiated fat cells
ACK: Ammonium-Chloride-Potassium
PEG 300: Polyethylene glycol 300
ALP: Alkaline phosphatase
C/EBPβ: CCAAT/enhancer binding protein beta
PPARγ: Peroxisome proliferator-activated receptor gamma
CD13: Aminopeptidase N
Runx2: RUNX Family Transcription Factor 2
Smad3: Mothers against decapentaplegic homolog 3
Col2a1: Collagen Type II Alpha 1 Chain
Sox9: SRY-Box Transcription Factor 9
ATP: Adenosine triphosphate
NDUFA9: NADH:Ubiquinone Oxidoreductase Subunit A9
UQCRC2: Ubiquinol-Cytochrome C Reductase Core Protein 2
VDAC: Voltage-dependent anion channel
CD81: Cluster of Differentiation 81
TNF-α: Tumor Necrosis Factor-α
IL-6: Interleukin 6
RIP1: Receptor-interacting serine/threonine-protein 1
CEBPA: CCAAT/enhancer-binding protein alpha
MCP-1: Monocyte chemoattractant protein-1
Esrrb: Estrogen Related Receptor Beta
Sox2: Sex determining region Y-box 2
Smad9: Mothers against decapentaplegic homolog 9

### REFERENCES

The disclosures of the following references are incorporated by reference
[1] Q. A. Wang, A. Song, W. Chen, P. C. Schwalie, F. Zhang, L. Vishvanath, L. Jiang, R. Ye, M. Shao, C. Tao, R. K. Gupta, B. Deplancke, P. E. Scherer, Reversible Dedifferentiation of Mature White Adipocytes into Preadipocyte-like Precursors during Lactation, Cell Metab, 28 (2018) 282-288 e283.
[2] B. Gustafson, U. Smith, Activation of canonical wingless-type MMTV integration site family (Wnt) signaling in mature adipocytes increases beta-catenin levels and leads to cell dedifferentiation and insulin resistance, J Biol Chem, 285 (2010) 14031-14041.
[3] L. Bochet, C. Lehuede, S. Dauvillier, Y. Y. Wang, B. Dirat, V. Laurent, C. Dray, R. Guiet, I. Maridonneau-Parini, S. Le Gonidec, B. Couderc, G. Escourrou, P. Valet, C. Muller, Adipocyte-derived fibroblasts promote tumor progression and contribute to the desmoplastic reaction in breast cancer, Cancer Res, 73 (2013) 5657-5668.
[4] P. Bi, F. Yue, A. Karki, B. Castro, S. E. Wirbisky, C. Wang, A. Durkes, B. D. Elzey, O. M. Andrisani, C. A. Bidwell, J. L. Freeman, S. F. Konieczny, S. Kuang, Notch activation drives adipocyte dedifferentiation and tumorigenic transformation in mice, J Exp Med, 213 (2016) 2019-2037.
[5] S. E. Ross, N. Hemati, K. A. Longo, C. N. Bennett, P. C. Lucas, R. L. Erickson, O. A. MacDougald, Inhibition of adipogenesis by Wnt signaling, Science, 289 (2000) 950-953.
[6] C. Christodoulides, C. Lagathu, J. K. Sethi, A. Vidal-Puig, Adipogenesis and WNT signalling, Trends Endocrinol Metab, 20 (2009) 16-24.
[7] L. Choy, J. Skillington, R. Derynck, Roles of autocrine TGF-beta receptor and Smad signaling in adipocyte differentiation, J Cell Biol, 149 (2000) 667-682.
[8] W. P. Cawthorn, F. Heyd, K. Hegyi, J. K. Sethi, Tumour necrosis factor-alpha inhibits adipogenesis via a beta-catenin/TCF4(TCF7L2)-dependent pathway, Cell Death Differ, 14 (2007) 1361-1373.
[9] H. Sugihara, N. Yonemitsu, S. Miyabara, K. Yun, Primary cultures of unilocular fat cells: characteristics of growth in vitro and changes in differentiation properties, Differentiation, 31 (1986) 42-49.
[10] M. Guo, A. F. Pegoraro, A. Mao, E. H. Zhou, P. R. Arany, Y. Han, D. T. Burnette, M. H. Jensen, K. E. Kasza, J. R. Moore, F. C. Mackintosh, J. J. Fredberg, D. J. Mooney, J. Lippincott-Schwartz, D. A. Weitz, Cell volume change through water efflux impacts cell stiffness and stem cell fate, Proc Natl Acad Sci USA, 114 (2017) E8618-E8627.
[11] Y. Li, A. S. Mao, B. R. Seo, X. Zhao, S. K. Gupta, M. Chen, Y. L. Han, T. Y. Shih, D. J. Mooney, M. Guo, Compression-induced dedifferentiation of adipocytes promotes tumor progression, Sci Adv, 6 (2020) eaax5611.
[12] D. Mohammed, C. Y. Park, J. J. Fredberg, D. A. Weitz, Tumorigenic mesenchymal clusters are less sensitive to moderate osmotic stresses due to low amounts of junctional E-cadherin, Sci Rep, 11 (2021) 16279.
[13] J. F. Shen, A. Sugawara, J. Yamashita, H. Ogura, S. Sato, Dedifferentiated fat cells: an alternative source of adult multipotent cells from the adipose tissues, Int J Oral Sci, 3 (2011) 117-124.
[14] M. Rosina, V. Ceci, R. Turchi, L. Chuan, N. Borcherding, F. Sciarretta, M. Sanchez-Diaz, F. Tortolici, K. Karlinsey, V. Chiurchiu, C. Fuoco, R. Giwa, R. L. Field, M. Audano, S. Arena, A. Palma, F. Riccio, F. Shamsi, G. Renzone, M. Verri, A. Crescenzi, S. Rizza, F. Faienza, G. Filomeni, S. Kooijman, S. Rufini, A. A. F. de Vries, A. Scaloni, N. Mitro, Y. H. Tseng, A. Hidalgo, B. Zhou, J. R. Brestoff, K. Aquilano, D. Lettieri-Barbato, Ejection of damaged mitochondria and their removal by macrophages ensure efficient thermogenesis in brown adipose tissue, Cell Metab, 34 (2022) 533-548 e512.
[15] A. Maeda, B. Fadeel, Mitochondria released by cells undergoing TNF-alpha-induced necroptosis act as danger signals, Cell Death Dis, 5 (2014) e1312.
[16] H. Xu, J. K. Sethi, G. S. Hotamisligil, Transmembrane tumor necrosis factor (TNF)-alpha inhibits adipocyte differentiation by selectively activating TNF receptor 1, J Biol Chem, 274 (1999) 26287-26295.
[17] Q. Zhu, Y. Zhu, C. Hepler, Q. Zhang, J. Park, C. Gliniak, G. H. Henry, C. Crewe, D. Bu, Z. Zhang, S. Zhao, T. Morley, N. Li, D. S. Kim, D. Strand, Y. Deng, J. J. Robino, O. Varlamov, R. Gordillo, M. G. Kolonin, C. M. Kusminski, R. K. Gupta, P. E. Scherer, Adipocyte mesenchymal transition contributes to mammary tumor progression, Cell Rep, 40 (2022) 111362.
[18] J. A. Nicolas-Avila, A. V. Lechuga-Vieco, L. Esteban-Martinez, M. Sanchez-Diaz, E. Diaz-Garcia, D. J. Santiago, A. Rubio-Ponce, J. L. Li, A. Balachander, J. A. Quintana, R. Martinez-de-Mena, B. Castejon-Vega, A. Pun-Garcia, P. G. Traves, E. Bonzon-Kulichenko, F. Garcia-Marques, L. Cusso, A. G. N, A. Gonzalez-Guerra, M. Roche-Molina, S. Martin-Salamanca, G. Crainiciuc, G. Guzman, J. Larrazabal, E. Herrero-Galan, J. Alegre-Cebollada, G. Lemke, C. V. Rothlin, L. J. Jimenez-Borreguero, G. Reyes, A. Castrillo, M. Desco, P. Munoz-Canoves, B. Ibanez, M. Torres, L. G. Ng, S. G. Priori, H. Bueno, J. Vazquez, M. D. Cordero, J. A. Bernal, J. A. Enriquez, A. Hidalgo, A Network of Macrophages Supports Mitochondrial Homeostasis in the Heart, Cell, 183 (2020) 94-109 e123.
[19] Z. Zhang, M. Shao, C. Hepler, Z. Zi, S. Zhao, Y. A. An, Y. Zhu, A. L. Ghaben, M. Y. Wang, N. Li, T. Onodera, N. Joffin, C. Crewe, Q. Zhu, L. Vishvanath, A. Kumar, C. Xing, Q. A. Wang, L. Gautron, Y. Deng, R. Gordillo, I. Kruglikov, C. M. Kusminski, R. K. Gupta, P. E. Scherer, Dermal adipose tissue has high plasticity and undergoes reversible dedifferentiation in mice, J Clin Invest, 129 (2019) 5327-5342.
[20] B. A. Shook, R. R. Wasko, O. Mano, M. Rutenberg-Schoenberg, M. C. Rudolph, B. Zirak, G. C. Rivera-Gonzalez, F. Lopez-Giraldez, S. Zarini, A. Rezza, D. A. Clark, M. Rendl, M. D. Rosenblum, M. B. Gerstein, V. Horsley, Dermal Adipocyte Lipolysis and Myofibroblast Conversion Are Required for Efficient Skin Repair, Cell Stem Cell, 26 (2020) 880-895 e886.

What is claimed is:

1. A method for inducing dedifferentiation of adipocytes, comprising subjecting one or more adipocytes to a hypertonic solution; and inducing release of mitochondrial extracellular vesicles (MEVs) from the one or more adipocytes to an extracellular environment, wherein released MEVs enhance the secretion of a series of inflammatory genes from the one or more adipocytes, and wherein the series of inflammatory genes activates the Wnt/β-catenin signaling pathway, thereby driving adipocyte dedifferentiation.

2. The method of claim 1, wherein the series of inflammatory genes comprise TNF-α, IL-6, RIP1, CEBPA, and MCP-1.

3. The method of claim 1, wherein the one or more adipocytes comprise primarily isolated adipocytes, adipocytes derived from induced pluripotent stem cells (iPSCs) or embryonic stem cells (ESCs), adipocytes from different anatomical locations.

4. The method of claim 1, wherein the one or more adipocytes comprise 3T3-L1 or stromal vascular fraction (SVF)-derived adipocytes.

5. The method of claim 1, wherein the adipocyte count of the one or more adipocytes is decreased by at least 3.5 times following hypertonic treatment.

6. The method of claim 5, wherein the hypertonic treatment results in reductions of 40-60% in the expression of adipogenic markers comprising C/EBPβ, PPAR-γ, and adiponectin.

7. The method of claim 5, wherein the hypertonic treatment results in increase of 10-20% in the expression of surface markers associated with dedifferentiated adipocytes.

8. The method of claim 1, wherein the hypertonic solution is formulated to have a hypertonic pressure compared to the surrounding environment, and the hypertonic solution comprises a culture medium and 2% PEG 300.

9. The method of claim 8, wherein the hypertonic solution further comprises an additive including a preservative, a stabilizer, or a medication.

10. The method of claim 1, wherein the one or more adipocytes treated with the hypertonic solution exhibit a capability for osteogenic and chondrogenic re-differentiation.

11. The method of claim 1, further comprising adding a small molecule compound to mitigate apoptosis of the one or more adipocytes induced by hypertonic treatment.

12. The method of claim 11, wherein the small molecule compound comprises 2-amino-4-(3,4-(methylenedioxy)benzylamino)-6-(3-methoxyphenyl)pyrimidine.

13. The method of claim 11, wherein the one or more adipocytes exhibit a decrease in the expression of adipogenic genes, coupled with concurrent elevations in the expression of marker genes.

14. The method of claim 13, wherein the adipogenic genes comprise C/EBPβ, PPAR-γ, and adiponectin.

15. The method of claim 13, wherein the marker genes comprise Smad 9, Esrrb, and Sox2.

16. The method of claim 11, wherein the one or more adipocytes exhibit a capability for osteogenic and chondrogenic re-differentiation.
